(19) United States
(12) Patent Application Publication
(10) Pub. No.: US 2020/0179530 A1
(43) Pub. Date: Jun. 11, 2020

(54) TARGETED DELIVERY OF THERAPEUTIC AGENTS AND IMAGING REAGENTS TO PANCREATIC CANCER CELLS

(71) Applicant: UNIVERSITY OF MIAMI, Miami, FL (US)

(72) Inventors: Camillo Ricordi, Miami, FL (US); Giacomo Lanzoni, Miami, FL (US)

(21) Appl. No.: 16/302,543

(22) PCT Filed: May 19, 2017
(86) PCT No.: PCT/US17/33591

§ 371 Date: Nov. 16, 2018

Related U.S. Application Data
(60) Provisional application No. 62/338,908, filed on May 19, 2016.

(51) Int. Cl. A61K 47/68; A61K 39/395; A61K 49/00; A61K 9/00

(52) U.S. Cl. CPC A61K 47/6898; A61K 39/3955; A61K 9/0019; A61K 49/00; A61K 39/39558

(57) ABSTRACT

The invention is directed to a method for cancer treatment and prevention, specifically, a method of targeting and delivering therapeutic agents and/or imaging reagent to cells expressing Purinergic Receptor P2Y G-Protein Coupled 1 (P2RY1).

### CROSS-REFERENCE TO RELATED APPLICATIONS AND INCORPORATION BY REFERENCE OF MATERIAL SUBMITTED ELECTRONICALLY

This application claims priority to U.S. Provisional Patent Application No. 62/338,908, filed May 19, 2016, the disclosure of which is hereby incorporated by reference in its entirety.

This application contains, as a separate part of the disclosure, a Sequence Listing in computer-readable form which is incorporated by reference in its entirety and identified as follows: Filename: 50472A_Seqlisting.txt; Size: 39,824 bytes; Created: May 19, 2017.

### FIELD OF THE INVENTION

The invention relates, in general, to materials and methods for delivering an agent to cells expressing Pancreatic and Duodenal Homeobox 1 (PDX1), cells expressing Purinergic Receptor P2Y G-Protein Coupled 1 (P2RY1), and pancreatic cancer cells.

### BACKGROUND

Cancer is a group of diseases characterized by the uncontrolled growth and spread of abnormal cells. In the early part of the next century, cancer is predicted to become the leading cause of death. Pancreatic adenocarcinoma is among the most lethal common cancers; it is frequently diagnosed at an advanced stage and is resistant to current therapies (Ryan et al., NEJM 371:1039-49 (2014)). Fewer than 20% of patients present with a sufficiently localized tumor to allow radical treatment. As a result, the 5-year survival of patients with pancreatic adenocarcinoma is lower than 5% (Hidalgo M. N Engl J Med. 362:1605-17 (2010)). According to statistics from the American Cancer Society, approximately 53,000 Americans are expected to be diagnosed with pancreatic cancer and approximately 41,000 of those will die of the disease in 2016 (Cancer Facts & Figures 2016, American Cancer Society). There is an urgent need for the development of effective therapies for cancer, particularly for those cancers that fail to respond to current therapy, such as pancreatic adenocarcinoma.

### SUMMARY

The disclosure provides materials and methods for delivering agents to cells that express Pancreatic and Duodenal Homeobox 1 (PDX1) by targeting Purinergic Receptor P2Y G-Protein Coupled 1 (P2RY1) expressed on the cell surface. In one aspect of the invention, the materials and methods are used to deliver an agent, such as a cytotoxic agent or an imaging agent, to cancer cells.

The disclosure provides a method of delivering an agent to a cell expressing PDX1. The method comprises contacting the cell with (i) a targeting moiety that binds Purinergic Receptor P2Y G-Protein Coupled 1 (P2RY1) and (ii) an agent comprising a binding moiety that binds the targeting moiety.

In some embodiments, the targeting moiety is an anti-P2RY1 antibody or antigen-binding fragment thereof, aptamer, or P2RY1 ligand. When the targeting moiety is an anti-P2RY1 antibody or antigen-binding fragment thereof, the targeting moiety is optionally conjugated to avidin, streptavidin, or variants of avidin or streptavidin, such as an oxidized variant of avidin (e.g., AvidinOX®). In other embodiments, when the targeting moiety is an anti-P2RY1 antibody or antigen-binding fragment, the targeting moiety is optionally conjugated to biotin. In some embodiments the targeting moiety that binds P2RY1 is bound to an agent and/or to additional targeting moieties via covalent bond (sharing of electron pair) or non-covalent bond (i.e., hydrogen bond, ionic bond, van der Waals interaction, or hydrophobic bond).

Optionally, the method further comprises contacting the cell with (iii) a targeting moiety that binds a member of the Carcinoembryonic antigen (CEA) family (such as, for example, Carcinoembryonic antigen-related cell adhesion molecule 8 (CEACAM8) or Carcinoembryonic antigen-related cell adhesion molecule 6 (CEACAM6)) or a targeting moiety that binds Mesothelin (MSLN). The targeting moiety that binds a member of the CEA family (e.g., CEACAM8 or CEACAM6) or the targeting moiety that binds MSLN is, in various embodiments, an antibody or antigen-binding fragment thereof, aptamer, or ligand. In some aspects, the targeting moiety is conjugated to biotin. In other aspects, the targeting moiety that binds a member of the CEA family and/or the targeting moiety that binds MSLN optionally is conjugated to avidin, streptavidin, or a variant thereof, such as an oxidized variant of avidin (e.g. AvidinOX®).

Optionally, the method further comprises contacting the biotin-conjugated targeting moieties with binding moieties, such as streptavidin, or avidin, or their variants (e.g. AvidinOX®, an oxidized variant of avidin), or variants covalently or non-covalently bound to agents. Optionally, the method further comprises contacting the targeting moieties or binding moieties with agents conjugated with biotin.

Exemplary agents for use in the context of the method include, but are not limited to, a cytotoxic agent, an immune mediator, a growth factor, an immunosuppressant, a radioactive substance, a metal, a cell, a hormone, an extracellular matrix component, a cell-adhesion molecule, a biomaterial, or an imaging agent (e.g., a fluorochrome). In various embodiments, the agent comprises a binding moiety that binds the targeting moiety. For example, in some aspects, the (i) targeting moiety is a P2RY1 antibody or antigen-binding fragment thereof conjugated to biotin, (ii) the agent-binding moiety is an oxidized variant of avidin, and (iii) the agent is (a) yttrium-90 1,4,7,10-tetraazacyclododecane-1,4,7,10-tetraacetic acid conjugated to biotin (⁹⁰Y-DOTA-biotin) or (b) Fluorine-18-labelled biotin (¹⁸F-biotin). In another example, the (i) targeting moiety is a P2RY1 antibody conjugated to biotin, the (ii) agent-binding moiety is an oxidized variant of avidin, and the (iii) agent is ⁹⁰Y-DOTA-biotin, wherein the (ii) agent-binding moiety and (iii) agent are combined to form an unsaturated complex. In some embodiments, the agent is covalently bound to an agent-binding moiety or the targeting moiety. In other embodiments, the agent has a non-covalent interaction (e.g., hydrogen bonds, ionic bonds, van der Waals interactions, and hydrophobic bonds) with an agent-binding moiety and/or with the targeting moiety.

In one aspect, the (i) targeting moiety that binds P2RY1 and (ii) agent are given simultaneously (optionally also with an agent-binding moiety), such as in the same formulation. If a (iii) targeting moiety that binds a member of the CEA family or a targeting moiety that binds MSLN is also utilized, the additional targeting moiety may be given simultaneously with (i) the targeting moiety that binds P2RY1 and/or (ii) the agent. Alternatively, the (i) targeting moiety that binds P2RY1 and (ii) agent (optionally with an agent-binding moiety) can be administered in separate formulations and administered concurrently (near in time), or the (i) targeting moiety is administered first and the (ii) agent (optionally with an agent-binding moiety) is administered later in time (e.g., at least one day following administration of the targeting agent). Similarly, if a (iii) targeting moiety that binds a member of the CEA family and/or a targeting moiety that binds MSLN also is utilized, the additional targeting moiety may be given in a formulation separate from (i) the targeting moiety that binds P2RY1 and/or (ii) agent (optionally with an agent-binding moiety). The strategy where the targeting moiety is given first, and the binding moiety and agent are given at a later time, is referred to as “pretargeting.”

In some embodiments, the agent (optionally with the agent-binding moiety) is combined to the targeting moiety that binds P2RY1 prior to administration to the subject. For example, the disclosure provides a method of delivering an agent to a cell expressing PDX1, wherein the method comprises contacting the cell with a targeting moiety that binds P2RY1 conjugated to the agent. In some cases, the agent does not comprise (or is not attached to) a separate binding moiety. Optionally, the agent is further conjugated to a targeting moiety that binds a member of the CEA family and/or a targeting moiety that binds MSLN.

The method described herein is suitable for delivering an agent to any cell that expresses PDX1 and P2RY1, such as pancreatic PDX1-expressing cells and pancreatic adenocarcinoma PDX1-expressing cells. In some embodiments the cell is a pancreatic cancer cell, a pancreatic islet beta cell, a pancreatic duct cell, or a pancreatic progenitor cell. Optionally, the method further comprises determining expression of PDX1 and/or expression of P2RY1 in a sample of the cells prior to administration of targeting moiety and/or agent. The method optionally comprises determining expression of members of the CEA family (e.g., CEACAM6, CEACAM8) and/or expression of MSLN in a sample of the cells prior to administration of targeting moiety and/or agent. The cell may be contacted in vitro or in vivo with the targeting moiety (or targeting moieties) and agent. When contacting the cell in vivo, the targeting moiety that binds P2RY1 and agent (and optionally the targeting moiety that binds a member of the CEA family and/or a targeting moiety that binds MSLN) are delivered intravenously, by intra-pancreatic artery infusion, or by intra-pancreatic duct infusion in various aspects of the disclosed method.

The disclosure further provides a method of treating pancreatic cancer in a subject. The method comprises administering to the subject a targeting moiety that binds P2RY1, as described herein, and a cytotoxic agent that couples to the targeting moiety. The pancreatic cancer cells express P2RY1. In various aspects, the pancreatic cancer cells express a member of the CEA family and/or MSLN, and the method also comprises administering a targeting moiety that binds a member of the CEA family and/or a targeting moiety that binds MSLN. Optionally, expression of P2RY1, CEA, and/or MSLN is identified in a sample from the subject prior to treatment. In some embodiments, tumor size, tumor burden, and/or metastasis is reduced in the subject. The disclosure also provides a method of detecting a cell expressing PDX1. The method comprises (a) administering to the subject (i) the targeting moiety that binds P2RY1 and (ii) an imaging agent that couples to the targeting moiety, and (b) detecting the imaging agent. In various aspects, the method further comprises administering to the subject a targeting moiety that binds a member of the CEA family and/or a targeting moiety that binds MSLN, and the imaging agent couples to the targeting moiety (or moieties).

Aspects of the disclosure that are described as methods can alternatively be described as uses. For instance, a related aspect of the disclosure is the use of (i) a targeting moiety that binds P2RY1 and (ii) an agent comprising a binding moiety that binds the targeting moiety for delivering the agent to a cell expressing PDX1. The disclosure further provides (a) use of a targeting moiety that binds P2RY1 conjugated to an agent for delivering the agent to a cell expressing PDX1, (b) use of the targeting moiety that binds P2RY1 and a cytotoxic agent that couples to the targeting moiety in a method of treating cancer (such as pancreatic cancer), and (c) use of the targeting moiety that binds P2RY1 and an imaging agent that couples to the targeting moiety for detecting a cell expressing PDX1. The disclosure further provides the targeting moiety, agent, and binding moieties described herein for use in, e.g., delivering the agent to a cell expressing PDX1, a method of treating cancer in a subject in need thereof, and for detecting a cell expressing PDX1. The disclosure further provides a composition (e.g., a pharmaceutical composition) comprising the targeting moiety and/or agent (optionally comprising the binding moiety), optionally for use in any of the methods described herein.

Additional aspects of the invention will be apparent from the Detailed Description, Figures, and Examples set forth below. Section headings are for the convenience of reading and not intended to be limiting per se. The entire document is intended to be viewed as a unified disclosure, and it should be understood that all combinations of features described herein are contemplated, even if the combination of features are not found together in the same sentence, or paragraph, or section of this document. It will be understood that any feature of the methods described herein can be deleted, combined with, or substituted for, in whole or part, any other feature described herein.

### BRIEF DESCRIPTION OF THE FIGURES

FIG. 1A and FIG. 1B are bar graphs illustrating relative gene expression (v/s islets; y-axis) of PDX1 and P2RY1 (FIG. 1A) or DRD-1 (FIG. 1B) in 24 pancreatic subpopulations sorted via fluorescence activated cell sorting (FACS). Two sets of bars are present for each sorted pancreatic subpopulation; the bar on the left of each pair corresponds to PDX1, while the bar on the right of each pair corresponds to P2RY1 (FIG. 1A) or DRD-1 (FIG. 1B). The dataset generated by Dorrell et al. (Diabetologia. 2011 November; 54(11):2832-44. doi: 10.1007/s00125-011-2283-5. Epub 2011 Sep. 1) was analyzed. The analysis enabled the identification of the surface protein P2RY1 as a marker with an expression profile similar to that of PDX1 throughout cell types. DRD-1 was observed to have opposite expression levels, when compared to PDX1, throughout cell types.

FIG. 2 is an illustration of an embodiment of the invention. Biotinylated antibodies that target the extracellular portion of P2RY1 are utilized as targeting moieties, whereas unsaturated complexes of avidin with ⁹⁰Y-DOTA-biotin are utilized as binding moieties. After administration to a subject via, e.g., infusion (panel 1), the targeting moieties contact P2RY1 (panel 2). Radioactive binding moieties are subsequently infused (3) and form a complex with the biotinylated antibodies on the surface of target cells (4). In an alternative embodiment, targeting moieties are composed of unsaturated complexes of biotinylated antibodies and AvidinOX. After infusion, the targeting moieties bind P2RY1. Hours or days after infusion, a radioactive binding moiety such as ⁹⁰Y-DOTA-biotin is administered and form complexes with the targeting moiety on the surface of target cells.

FIGS. 3A-3D: Quantification of P2RY1 (FIG. 3A), CEACAM8 (FIG. 3B), CEACAM6 (FIG. 3C), and MSLN (FIG. 3D) proteins in immunohistochemical stainings of healthy and cancerous pancreatic tissue samples (tissue microarrays). Tissue microarrays with human healthy and cancerous pancreatic sections were incubated with antibodies anti-P2RY1, anti-CEACAM8 (CD66b), anti-CEACAM6 (CD66c), or anti-MSLN, and subsequently stained via immunohistochemical (IHC) method. The resulting stained microarrays were scanned. The signal intensity in the red channel (immunohistochemical, IHC stain) was quantified for each sample, and normalized based on the surface of the tissue section. Each dot in the scatter plot represents the IHC signal intensity (Arbitrary Units, AU (y-axis)) for a particular tissue sample (x-axis). The mean is shown with a longer bar, along with the standard error of the mean (error bars), to determine how precisely the mean of the sample estimates the population mean. The mean values were found to be higher in pancreatic cancer samples. The variances of the values obtained in healthy pancreas versus pancreatic cancer samples were compared by using the F test. The differences between variances were found to be highly significant for CEACAM8 and MSLN. A subset of pancreatic cancer samples appears to express the indicated proteins at high levels.

FIG. 4: Pretargeting with biotinylated anti-P2RY1 antibodies enables the specific accumulation of fluorescently labeled streptavidin. The experiment was conducted on live adherent cell cultures of human pancreatic exocrine cells containing P2RY1-expressing cells, and of human pancreatic mesenchymal cells that do not express P2RY1. Anti-P2RY1 antibodies were added to the culture medium, incubated with live cells for 1 hour at 37° C., and subsequently removed with the supernatant with multiple washes in PBS (an approach termed “pretargeting”). Fluorescently labeled streptavidin was added to the live cell cultures. Cell cultures were subsequently imaged with an Essen Incucyte Zoom instrument in brightfield and red fluorescence illumination to detect the accumulation of fluorescence signal from labeled streptavidin. Images were acquired every five minutes. Normalized fluorescence integrated intensity is presented in the graph (y-axis) as fold change versus the value at Time 0. An accumulation of fluorescently labeled streptavidin on pancreatic exocrine cells was observed, indicating accumulation in correspondence to biotinylated anti-P2RY1 antibodies. The results demonstrate that pretargeting with a targeting agent that binds P2RY1 (e.g., biotinylated anti-P2RY1 antibodies) enables accumulation of streptavidin in live PDX1-expressing pancreatic exocrine cells.

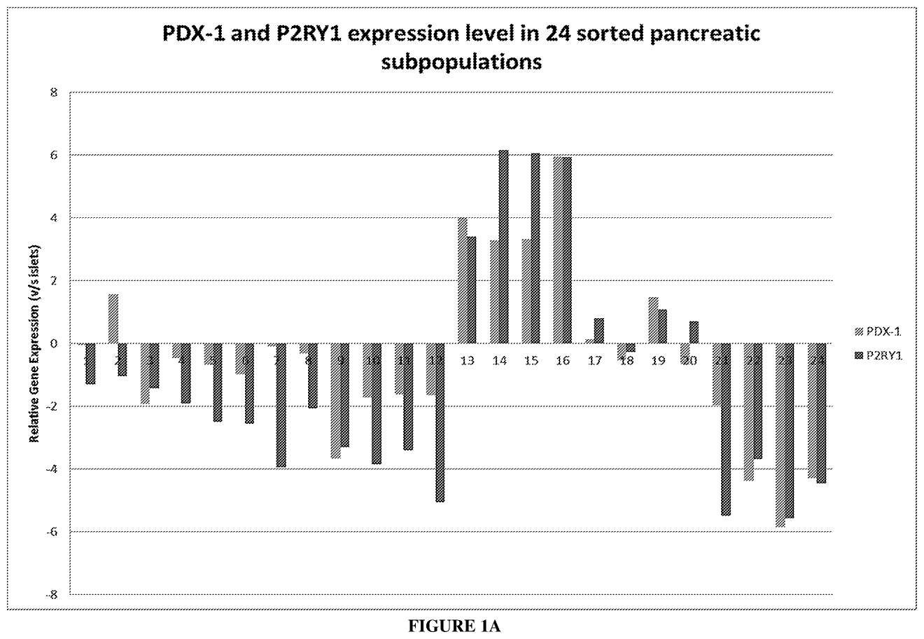

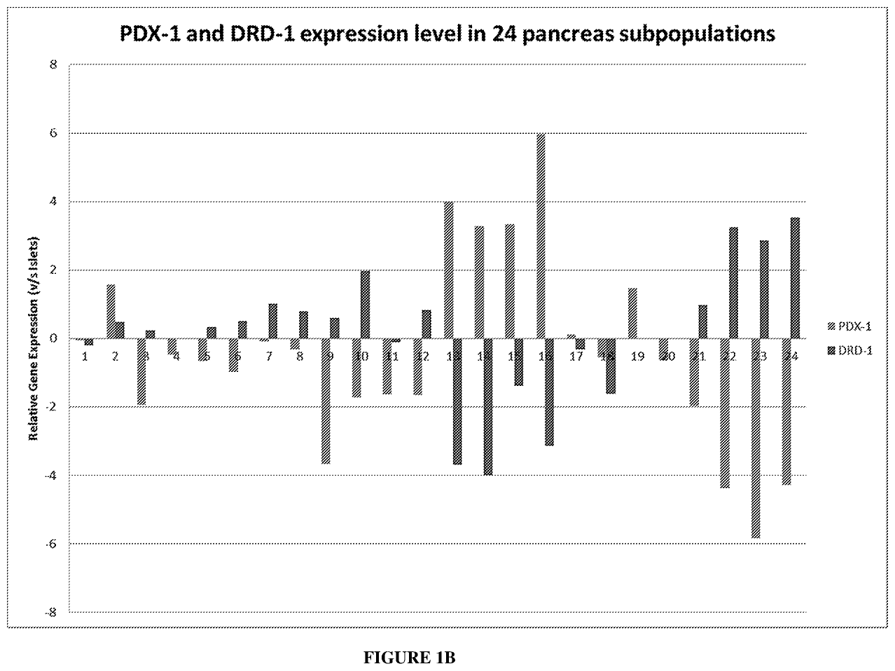

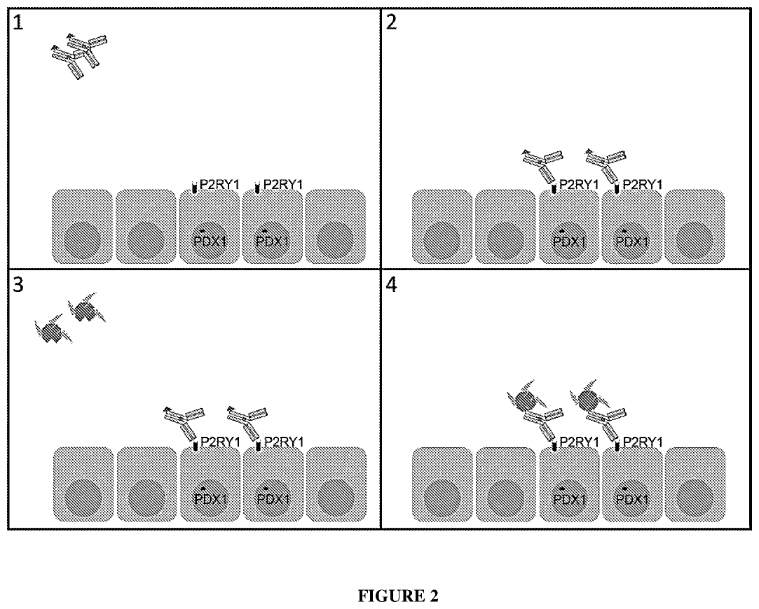

### DETAILED DESCRIPTION

The disclosure provides, in various aspects, methods of targeted delivery of agents (e.g., therapeutic agents and/or imaging agents) to cells expressing PDX1, such as a subset of pancreatic cells, pancreatic adenocarcinoma cells and other cancer cell types. The methods of the disclosure exploit the unexpected discovery that cells expressing the transcription factor PDX1 also express the cell surface marker P2RY1 at sufficiently high levels to allow targeted delivery to the desired cell. Thus, in various embodiments, the invention includes a method of delivering an agent to a cell expressing PDX1, the method comprising contacting the cell with a targeting moiety that binds P2RY1 and an agent, which is coupled to the targeting moiety or which comprises a binding moiety that binds the targeting moiety to create a complex, thereby delivering the agent to PDX1-expressing cells.

### Pancreatic and Duodenum Homeobox 1 (PDX1)

Pancreatic and Duodenum Homeobox 1 (PDX1) is a transcription factor that is expressed in a variety of cell types, including beta (β) and delta (δ) cells of the islets of Langerhans, pancreatic ductal cells, pancreatic progenitor cells, and duodenal cells (Mckinnon and Docherty, Diabetologia 44: 1203-14 (2001)). PDX1 is a key regulator of pancreatic development, β-cell differentiation, normal β-cell function, and pancreatic cancer (Wu et al., Cancer Gene Therapy 21, 48-53 (2014)). Previous studies have demonstrated that PDX1 is overexpressed in pancreatic cancer, regulates pancreatic cancer cell lines, and is a potential therapeutic target for pancreatic cancer (Wu et al., Cancer Gene Therapy 21, 48-53 (2014). In addition, PDX1 expression in pancreatic ductal carcinomas has been reportedly associated with a poor prognosis (Park et al., Appl Immunohistochem Mol Morphol. 19(5): 444-449 (2011)). However, PDX1 is a transcription factor localized into the nucleus and, thus, it is not easily exploited for targeting purposes.

### Purinergic Receptor P2Y G-Protein Coupled 1 (P2RY1)

P2Y receptors are a family of plasma membrane-associated purinergic G-protein-coupled receptors (GPCRs) having seven transmembrane regions. In mammalian cells, eight P2Y-receptor subtypes have been cloned and identified as P2-receptors. P2RY1 and P2RY2 have been shown to be expressed in pancreas among other tissues (Coutinho-Silva et al., G. Mol. Cell. Endocrinol. 204:141-54 (2003). P2RY1 is further described in International Patent Publication Nos. WO 2001/090117 and WO 2003/101284.

The methods of the disclosure exploit the unexpected discovery that P2RY1 expression can serve as a surrogate for PDX1 expression. Cells expressing the transcription factor PDX1 at high levels were found to also express the surface marker P2RY1 at high levels. The expression of PDX1 and P2RY1 is characteristic of, e.g., certain pancreatic cell subsets (such as islet beta cells and pancreatic duct cells) and, notably, pancreatic tumor cells. P2RY1 represents a novel means of targeting PDX1-expressing cells.

### Mesothelin and Carcinoembryonic Antigen Family (CEACAM8 and CEACAM6)

Various aspects of the method comprise use of a targeting moiety that binds mesothelin (MSLN) in combination with the targeting moiety that binds P2RY1. Mesothelin is a 40 kDa protein found on mesothelial cells, as well as several tumors types (e.g., ovarian cancer and pancreatic adenocarcinoma). The protein is a glycosylphosphatidylinositol-anchored cell-surface protein. Mesothelin is further described in Chang et al., Proc Natl Acad Sci USA 93:136-140 (1996); Yamaguchi et al., J. Biol. Chem. 269:805-808 (1994); and U.S. Pat. No. 9,416,190, each of which are incorporated by reference, particularly with respect to the description of mesothelin protein and/or antibodies that bind mesothelin.

Various aspects of the method comprise use of a targeting moiety that binds a member of the Carcinoembryonic Antigen (CEA) family, such as carcinoembryonic antigen-related cell adhesion molecule 8 (CEACAM8) or carcinoembryonic antigen-related cell adhesion molecule 6 (CEACAM6), in combination with the targeting moiety that binds P2RY1. Members of the CEA family play a role in cell adhesion and are widely used as tumor markers in serum immunoassay determinations of carcinoma. CEACAM8 (also known as CD66b (Cluster of Differentiation 66b)) is a glycosylphosphatidylinositol-anchored membrane glycoprotein having a molecular weight of around 95 kDa. The protein is a product of the CEACAM8 gene (previously known as CGM6), and is associated with cell adhesion and migration. CEACAM8 is further described in Kuroki et al., J Leukoc Biol., 70(4):543-50 (2001); Yamenka et al., Biochem Biophys Res Commun., 219(3):842-7 (1996); and U.S. Pat. No. 8,309,091, which are incorporated by reference, particularly with respect to the description of CEACAM8 protein and/or antibodies that bind CEACAM8. CEACAM6 (also known as CD66c) is a GPI anchored cell surface glycoprotein having a molecular weight of around 37 kDa. The protein, a product of the CEACAM6 gene (previously known as NCA), plays a role in cell adhesion. CEACAM6 is further described in International Patent Publication No. WO/2016/150899, which is incorporated by reference, particularly with respect to the description of CEACAM6 protein and antibodies that bind CEACAM6.

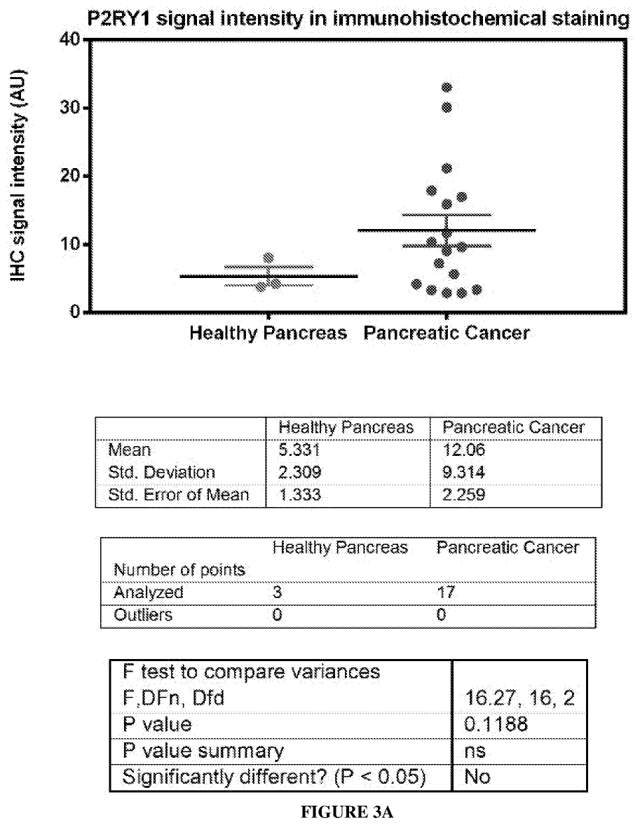

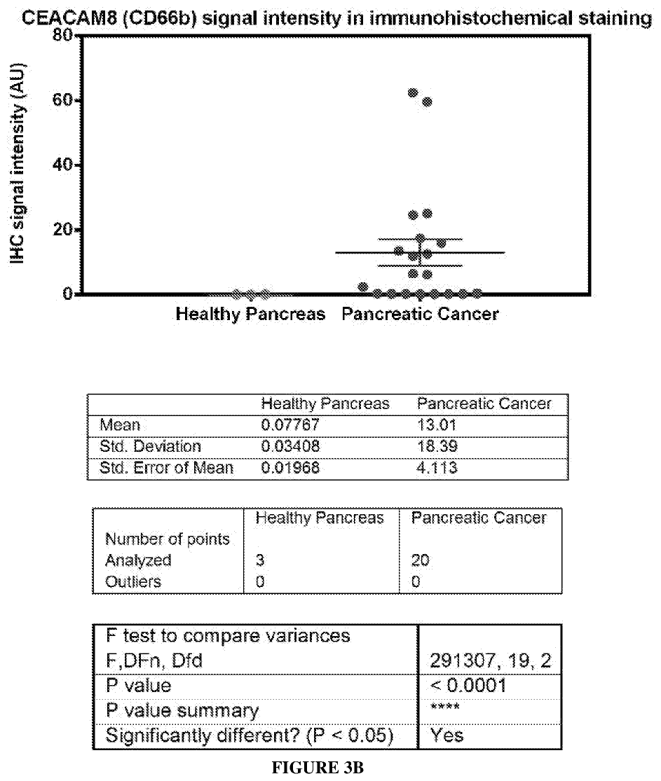

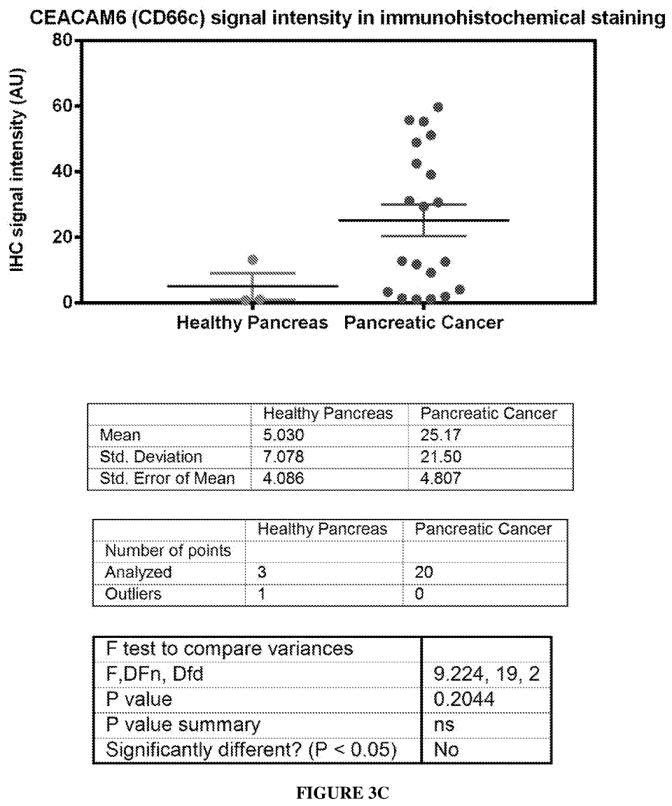

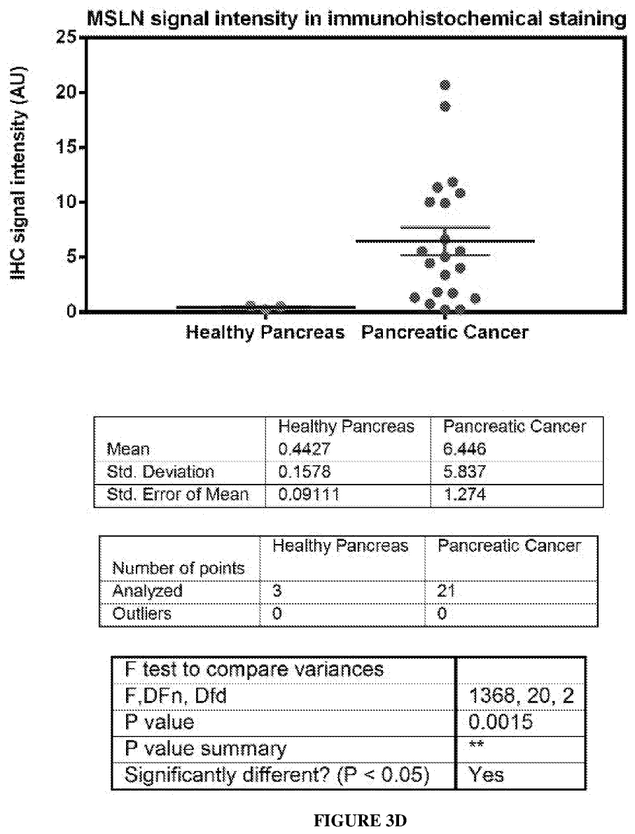

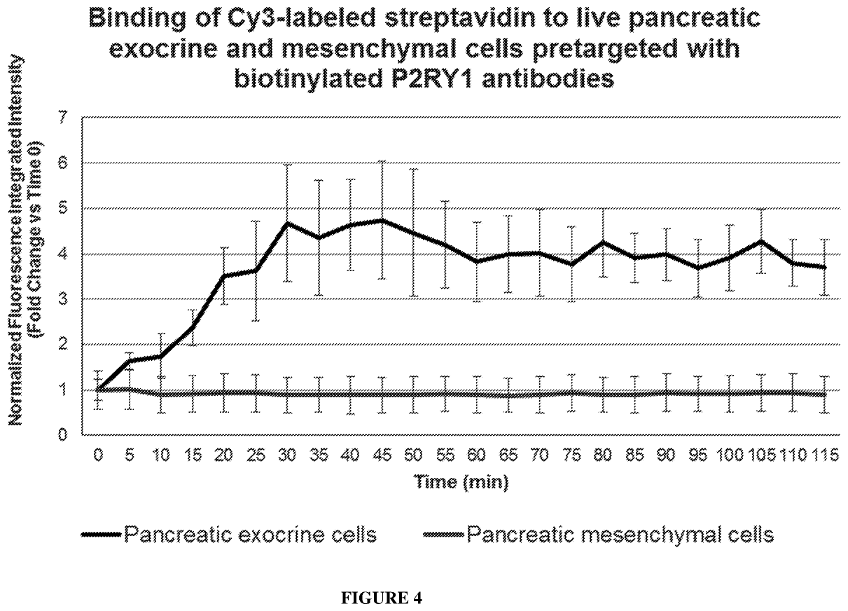

As demonstrated in FIGS. 3B-3D, MSLN and CEACAM8 are highly expressed in a subset of pancreatic cancer cells. In various aspects, use of a targeting moiety that binds MSLN, a targeting moiety that binds CEACAM8 (or other member of the CEA family), or both, in combination with the targeting moiety that binds P2RY1 enhances the avidity between the agent and a target cell via the binding to multiple cell surface targets through the different targeting moieties. Administering the targeting moiety that binds MSLN and/or the targeting moiety that binds CEACAM8 in combination with the targeting moiety that binds P2RY1 also, in various aspects, improves specificity of the method inasmuch as the agent is more strongly associated with cell types that display P2RY1 with MSLN and/or CEACAM8.

### Targeting Moieties

In various aspects, the method of the disclosure comprises contacting a cell or delivering to a subject a targeting moiety that binds P2RY1. In various aspects, the method of the disclosure further comprises contacting a cell or delivering to a subject a targeting moiety that binds CEACAM8 (or other member of CEA family, such as CEACAM6) and/or a targeting moiety that binds MSLN. Any suitable targeting moiety may be used in the context of the disclosure so long as the moiety binds P2RY1, CEACAM8 (or other member of CEA family, such as CEACAM6), or MSLN with sufficient affinity so as to allow delivery of the agent to the cell. For example, in various embodiments, the targeting moiety binds P2RY1, CEACAM8 (or other member of CEA family, such as CEACAM6), or MSLN with a binding affinity of greater than or equal to about 10⁻⁷, 10⁻⁸, 10⁻⁹, 10⁻¹⁰, 10⁻¹¹, or 10⁻¹² M. Optionally, the targeting moiety binds its target (P2RY1, CEACAM8 (or other member of CEA family, such as CEACAM6), or MSLN) with an equilibrium binding constant (K_{D}) that is less than the equilibrium binding constant observed for the binding of the targeting moiety to an unrelated protein (i.e., anti-target), preferably by a factor of 2 or less, such as by a factor of 5 or less or a factor of 10 or less. Binding affinity and specificity is determined using any of a number of assays, such as ELISA assays or surface plasmon resonance (e.g., BIACore).

Exemplary targeting moieties that bind P2RY1 include an anti-P2RY1 antibody or antigen-binding fragment thereof, an aptamer, or P2RY1 ligand. The method is, in various aspects, not dependent on a particular anti-P2RY1 antibody. An example of a commercially available anti-P2RY1 antibody is the rabbit polyclonal antibody for the extracellular portion of human P2RY1 (Alomone, catalog no.: APR-021). Using this antibody, PDX1 and P2RY1 immunofluorescence staining on sections of normal human pancreas established that the PDX1 and P2RY1 are very frequently expressed by the same cells. Additionally, PDX1 and P2RY1 immunofluorescence staining on sections of human pancreatic adenocarcinomas established that the two proteins are frequently co-expressed in a set of cells of epithelial morphology in these tumors.

Another example of a commercially available anti-P2RY1 antibody is the murine monoclonal antibody for human P2RY1 raised against partial recombinant P2RY1 protein (Abnova, catalog no.: H00005028-M01, clone 4C2; Creative Diagnostics, catalog no.: CABT-11849MH). Antibodies also may be generated using the full length or partial P2RY1 protein, the sequence of which is provided in SEQ ID NO: 1, using routine laboratory techniques, such as phage display technology, hybridoma technology, transgenic mouse technology and others. In various embodiments, the antibody is a monoclonal, human, humanized, and/or chimeric antibody.

Exemplary targeting moieties that bind members of the CEA family include an antibody or antigen-binding fragment thereof, an aptamer, or CEA ligand. CEA family antibodies include antibodies that target epitopes that are largely similar among different CEA proteins, such as Santa Cruz Biotechnology sc-59875 pan CEA (TET2). An exemplary aptamer that binds to CEA is presented in International Patent Publication number WO2013185241. Exemplary targeting moieties that bind CEACAM8 include an anti-CEACAM8 antibody or antigen-binding fragment thereof, an aptamer, or CEACAM8 ligand. CEACAM8 antibodies include BioLegend 305102 anti-human CD66b (clone G10F5). Exemplary targeting moieties that bind CEACAM6 include an anti-CEACAM6 antibody or antigen-binding fragment thereof, an aptamer, or CEACAM6 ligand. An exemplary targeting moiety is MBL Life Science—D028-3 Anti-CD66c (clone KOR-SA3544). Exemplary targeting moieties that bind MSLN include an anti-MSLN antibody or antigen-binding fragment thereof, an aptamer, or MSLN ligand. MSLN antibodies include, but are not limited to MORAb-009 (also known as amatuximab), m912, HN1, and Leica Biosystems Novocastra™ NCL-L-MESO (clone 5B2). See, e.g., Feng et al., Mol Cancer Ther 8:1113-1118, 2009; Ho et al., Int J Cancer 128:2020-2030, 2011; Hassan et al., Cancer Immun 7:20, 2007.

An antibody (e.g., anti-P2RY1 antibody, anti-CEACAM8 antibody, anti-CEACAM6 antibody, or anti-MSLN antibody) may comprise a complete antibody molecule, i.e., an immunoglobulin having full length heavy and light chains. The targeting moiety may alternatively be an antigen-binding fragment of an antibody (e.g., anti-P2RY1 antibody, anti-CEA antibody, anti-CEACAM8 antibody, anti-CEACAM6 antibody, or anti-MSLN antibody). Antibody fragments, which include F(ab′)₂, Fab, Fab′, Fv, Fc, and Fd fragments, retain the antigen binding site of the whole antibody and, therefore, bind to the same epitope. Antigen-binding fragments derived from an antibody can be obtained, for example, by proteolytic hydrolysis of the antibody, for example, pepsin or papain digestion of whole antibodies according to conventional methods. As an illustration, antibody fragments can be produced by enzymatic cleavage of antibodies with pepsin to provide a 5S fragment denoted F(ab′)₂. This fragment can be further cleaved using a thiol reducing agent to produce 3.5S Fab′ monovalent fragments. Optionally, the cleavage reaction can be performed using a blocking group for the sulfhydryl groups that result from cleavage of disulfide linkages. As an alternative, an enzymatic cleavage using papain produces two monovalent Fab fragments and an Fc fragment directly. These methods are described, for example, by Goldenberg, U.S. Pat. No. 4,331,647, Nisonoff et al., Arch. Biochem. Biophys. 89:230, 1960; Porter, Biochem. J. 73:119, 1959; Edelman et al., in Methods in Enzymology 1:422 (Academic Press 1967); and by Coligan at pages 2.8.1-2.8.10 and 2.10.-2.10.4. Other methods for cleaving antibodies, such as separating heavy chains to form monovalent light-heavy chain fragments (Fd), further cleaving of fragments, or other enzymatic, chemical, or genetic techniques may also be used, so long as the fragments bind to the antigen that is recognized by the intact antibody.

Aptamers are oligonucleotide or peptide molecules that bind to a target molecule. Methods of generating aptamers and assaying for protein binding are known in the art. See, e.g., (Sun et al., Molecular Therapy Nucleic Acids, 3:e182 (2014); Deng et al., Analytica Chimica Acta, 837, 1-15 (2014); Hoppe-Seyler, Current Molecular Medicine, 4, 529-538 (2004); Navani et al., Methods Mol Biol., 504, 399-415 (2009); U.S. Pat. No. 5,756,291; U.S. Patent Publication No. 2006/0105975; and International Patent Publication Nos. WO1992/014843 and WO2003/099840. The aptamer may be an RNA, a DNA, a modified nucleic acid, or peptide (or a mixture thereof). The aptamer may be in a linear or circular form.

Alternatively, the targeting moiety is a ligand for the target (e.g., a P2RY1 ligand, CEA ligand, CEACAM8 ligand, CEACAM6 ligand, or MSLN ligand). P2RY1 is a receptor for, e.g., extracellular ATP and ADP. Thus, the targeting moiety that binds P2RY1 is, in various aspects, ATP or ADP or a derivative thereof that binds P2RY1. Other P2RY1 ligands include, but are not limited to, MRS2500 tetraammonium salt, BPTU, BzATP triethylammonium salt, 2-Methylthio-ADP, MRS 2365, MRS 2179 tetrasodium salt, MRS 2279 and 2,2′-Pyridylisatogen tosylate.

In some embodiments, the targeting moiety is conjugated to the agent. Methods of conjugating peptides, peptides and small molecules, oligonucleotides and small molecules, etc. are well known in the art. See, e.g., Wang et al., J Med Chem., 56(6), 2527-39 (2013); Kim et al., PNAS, 110(44), 17796-17801 (2013); Thompson, “Molecular Diagnosis of Infectious Diseases,” Volume 94 of the series Methods in Molecular Medicine™, pp 255-265 (2004); Ahrens et al., Future Med Chem., 4(12), 1567-86 (2012). In various aspects, the targeting moiety or the agent (or binding moiety) comprises a functional group that facilitates conjugation to another moiety. Exemplary functional groups include, but are not limited to, isothiocyanate, isocyanate, acyl azide, NHS ester, sulfonyl chloride, aldehyde, epoxide, oxirane, carbonate, arylating agent, imidoester, carbodiimide, anhydride, alkyl halide derivatives (e.g., haloacetyl derivatives), maleimide, aziridine, acryloyl derivatives, arylating agents, thiol-disulfide exchange reagents (e.g., pyridyl disulfides or TNB thiol), diazoalkane, carboyldiimadazole, N,N′-Disuccinyl carbonate, N-Hydroxysuccinimidyl chloroformate, and hydrazine derivatives.

In one aspect, the targeting moiety is conjugated to streptavidin. An oxidized variant of avidin contemplated for use in the context of the invention is AvidinOX®. AvidinOX® refers to an avidin variant containing aldehyde groups, generated by ligand-assisted sugar oxidation of avidin by sodium periodate, maintains the capacity to bind biotin with very high affinity (Nucera et al., Int J Immunopathol Pharmacol. 25(1):239-46 (2012)). AvidinOX exhibits a half-life of weeks in tissue, whereas the half-life of native avidin is about two hours. AvidinOX is further described in Verdoliva, J. Biol. Chem., 285(12), 9090-9099 (2010); DeSantis et al., Cancer Biother Radiopharm., 25(2):143-8 (2010); as well as U.S. Pat. No. 8,562,947 and U.S. Patent Publication No. 20040134106, all of which are incorporated by reference herein with respect to their disclosures of oxidized avidin. Alternatively, the targeting moiety is conjugated to biotin. In various embodiments of the disclosure, the targeting moiety that binds P2RY1, as well as the targeting moiety that binds a member of the CEA family (e.g., CEACAM8, CEACAM6) and/or the targeting moiety that binds MSLN (if present), is biotinylated.

### Agents

The disclosure provides an improved method of delivering an agent to a cell expressing PDX1. Examples of agents suitable for use in the context of the method include, but are not limited to, chemotherapeutic/therapeutic agents (e.g., small molecules, immune mediators, growth factors, immunosuppressants, immunostimulants, hormones, enzymes, extracellular matrix components, cell-adhesion molecules, cells, or biomaterials) and imaging agents (e.g., positron-emitting moieties for Positron Emission Tomography, radionuclides, radionuclide-containing complex, fluorochromes, dyes, photosensitizers, metals such as contrast agents for Magnetic Resonance Imaging). Optionally, the agent comprises a binding agent that binds the targeting moiety.

Chemotherapeutic/therapeutic agents for use in conjunction with the method of the disclosure include, but are not limited to, alkylating agents (e.g., nitrogen mustards, such as mechlor-ethamine, cyclophosphamide, ifosfamide, melphalan and chlorambucil); nitrosoureas (e.g., carmustine (BCNU), lomustine (CCNU), and semustine (methyl-CCNU)); ethylenimines/methylmelamine (e.g., thriethylenemelamine (TEM), triethylene, thiophosphoramide (thiotepa), and hexamethylmelamine (HMM, altretamine)); alkyl sulfonates (e.g., busulfan); triazines (e.g., dacarbazine (DTIC)); antimetabolites, including folic acid analogs (e.g., methotrexate and trimetrexate); pyrimidine analogs (e.g., 5-fluorouracil, fluorodeoxyuridine, gemcitabine, cytosine arabinoside (AraC, cytarabine), 5-azacytidine, 2,2′-difluorodeoxycytidine)); purine analogs (e.g., 6-mercaptopurine, 6-thioguanine, azathioprine, 2′-deoxycoformycin (pentostatin), erythrohydroxynonyladenine (EHNA), fludarabine phosphate, and 2-chlorodeoxyadenosine (cladribine, 2-CdA)); antimitotic drugs (e.g., paclitaxel, vinca alkaloids including vinblastine (VLB), vincristine, vinorelbine, taxotere, estramustine, and estramustine phosphate); pipodophylotoxins (e.g., etoposide and teniposide); antibiotics (e.g., actimomycin D, daunomycin (rubidomycin), doxorubicin, mitoxantrone, idarubicin, bleomycins, plicamycin (mithramycin), mitomycinC, and actinomycin); enzymes (e.g., L-asparaginase); biological response modifiers (e.g., interferon-alpha, interleukin-2 (IL-2), Granulocyte-colony stimulating factor (G-CSF) and Granulocyte macrophage-colony stimulating factor (GM-CSF)); platinum coordination complexes (e.g., cisplatin and carboplatin, anthracenediones, hydroxyurea, methylhydrazine derivatives including N-methylhydrazine (MIH) and procarbazine); adrenocortical suppressants (e.g., mitotane and aminoglutethimide); hormones and adrenocorticosteroid antagonists (e.g., prednisone and equivalents, dexamethasone and aminoglutethimide); progestins (e.g., hydroxyprogesterone caproate, medroxyprogesterone acetate and megestrol acetate); estrogens (e.g., diethylstilbestrol and ethinyl estradiol equivalents); antiestrogens (e.g., tamoxifen); androgens (e.g., testosterone propionate and fluoxymesterone/equivalents); antiandrogens (e.g., flutamide, gonadotropin-releasing hormone analogs and leuprolide); and non-steroidal antiandrogens (e.g., flutamide).

In certain embodiments of the invention, P2RY1 is targeted for delivering an imaging agent to a cell expressing PDX1 to facilitate detection and imaging of target cells (e.g., a tumor). The method is useful for, e.g., monitoring or diagnosing the onset, development, progression and/or severity of a tumor as part of a clinical testing procedure, including in the context of methods for determining the efficacy of an anti-cancer therapy. By “imaging agent” is meant an agent that enables detection and/or visualization of a target cell or tissue. In various aspects, the imaging agent is a radionuclide, radioactive dye, or a radiolabelled compound. Imaging agents include, but are not limited to, enzymes (such as horseradish peroxidase, alkaline phosphatase, beta-galactosidase, or acetylcholinesterase); prosthetic groups (such as streptavidin/biotin and avidin/biotin); fluorescent materials (such as, e.g., Alexa Fluor 350, Alexa Fluor 405, Alexa Fluor 430, Alexa Fluor 488, Alexa Fluor 500, Alexa Fluor 514, Alexa Fluor 532, Alexa Fluor 546, Alexa Fluor 555, Alexa Fluor 568, Alexa Fluor 594, Alexa Fluor 610, Alexa Fluor 633, Alexa Fluor 647, Alexa Fluor 660, Alexa Fluor 680, Alexa Fluor 700, Alexa Fluor 750, umbelliferone, fluorescein, fluorescein isothiocyanate, rhodamine, dichlorotriazinylamine fluorescein, dansyl chloride or phycoerythrin); luminescent materials (such as luminol); bioluminescent materials (such as luciferase, luciferin, and aequorin); radioactive materials (such as radioactive iodine (₁₃₁I, ₁₂₅I, ₁₂₃I, and ₁₂₁I), radioactive carbon (₁₄C), radioactive sulfur (₃₅S), radioactive tritium (₃H), radioactive indium (₁₁₅In, ₁₁₃In, ₁₁₂In, and ₁₁₁In), radioactive technetium (₉₉Tc), radioactive thallium (₂₀1Ti), radioactive gallium (₆₈Ga, 67Ga), radioactive palladium (₁₉₃Pd), radioactive molybdenum (₉₉Mo), radioactive xenon (₁₃₃Xe), radioactive fluorine (¹⁸F), ₁₅₃Sm, ₁₇₇Lu, ₁₅₉Gd, ₁₄₉Pm, ₁₄₀La, ₁₇₅Yb, ₁₆₆Ho, ₉₀Y, ₄₇Sc, ₁₈₆Re, ₁₈₈Re, ₁₄₂Pr, ₁₀₅Rh, ₉₇Ru, ₆₈Ge, ₅₇Co, ₆₅Zn, ₈₅Sr, ₃₂P, ₁₅₃Gd, ₁₆₉Yb, ₅₁Cr, ₅₄Mn, ₇₅Se, ₆₄Cu, ₁₁₃Sn, and ₁₁₇Sn); positron emitting metals; and non-radioactive paramagnetic metal ions. Preferably, the imaging agent is suitable for in vivo imaging, although this is not required if detecting a target cell ex vivo. The detection step is optionally performed after a sufficient period of time to allow unbound imaging agent to clear from the target region and/or turnover of non-target cells.

In some embodiments, the agent is conjugated to the targeting moiety. For example, the targeting moiety is conjugated (or coupled) to the agent prior to contacting the cell or prior to administration to a subject. Optionally, the agent comprises (or is attached to) a binding moiety that binds the targeting moiety. A binding moiety is an agent that has affinity and specificity for the targeting moiety. Examples of binding agents include avidin or biotin, which forms complexes with biotin or avidin, respectively, attached to a targeting moiety.

The agent (or agent-binding moiety) and targeting moiety can be mixed prior to contacting the cell or administration to a subject, or the agent (or agent-binding moiety) and targeting moiety are separately applied to the cell or separately administered to the subject. Representative targeting moiety-agent combinations exploit biotin-avidin affinity. For example, in some embodiments, the targeting moiety is an antibody or antigen-binding fragment thereof conjugated to avidin, such as an oxidized version of avidin (e.g., AvidinOX®). The antibody or fragment thereof binds the cell surface marker P2RY1. The resulting complexes are stable for hours or days. The agent-binding moiety complex is a biotinylated agent (e.g., a biotinylated imaging agent, such as ¹⁸F-biotin, which is useful for positron emission tomography). The biotinylated agent is applied or administered subsequent to the avidin-targeting moiety complex, optionally after a period of time suitable for clearance of non-cell bound avidin-targeting moiety conjugates and/or non-target cell turnover. Biotin binds the avidin, effectively complexing the targeting moiety and agent (i.e., imaging agent) on P2RY1-expressing cells, allowing selective detection of PDX1 expressing cells. It will be appreciated that the description of targeting moieties that bind P2RY1 also apply to targeting moieties that bind MSLN and targeting moieties that bind CEACAM8.

In an alternative embodiment, the targeting moiety that binds P2RY1 (e.g., an anti-P2RY1 antibody or antigen-binding fragment thereof) is biotinylated. The agent-binding agent complex is an unsaturated complex of an oxidized variant of avidin (e.g., AvidinOX®). The agent is, in various aspects, a radionuclide therapeutic or imaging agent, such as ¹⁸F-biotin or ⁹⁰Y-DOTA-biotin (radioactive yttrium-90, complexed with the chelating agent DOTA and biotin), although other agents, such as those described herein, are also contemplated. Use of the unsaturated complex of an oxidized variant of avidin allows clustering of agents and targeting moieties to, e.g., enhance signal or therapeutic effect. In some aspects of the method, a targeting moiety that binds MSLN and/or a targeting moiety that binds CEACAM8 (or another member of the CEA family) also is administered. In these aspects, the targeting moiety that binds MSLN and/or the targeting moiety that binds CEACAM8 (or another member of the CEA family) is biotinylated, and also clusters with the agent and targeting moiety that binds P2RY1 via the oxidized variant of avidin.

In various embodiments comprising contacting a cell or administering to a subject an imaging agent, the method also comprises detecting the imaging agent, optionally in a manner that allows imaging of the cell or tissue of interest. In vivo imaging is achieved using, e.g., techniques that noninvasively produce images of an internal region of a subject. Examples of in vivo imaging techniques include, but are not limited to, are single-photon emission tomography (SPECT) and positron emission tomography (PET), both of which are well-known (see, e.g., “Emission Tomography: the Fundamentals of PET and SPECT”; 2004 Academic Press: Wernick and Aarsvold, Eds.). Compositions and methods relating to PET are described in, e.g., International Patent Publication No. WO 2006/071754, incorporated herein by reference.

### Expression Analysis

The method of the disclosure, in some embodiments, comprises determining expression of PDX1 and/or P2RY1 in a sample of the cells. In various aspects, the method comprises determining expression of one or more members of the CEA family (e.g. CEACAM8, CEACAM6), and/or MSLN. Expression can be detected by any suitable procedure, such as those available in the art. Expression of is detected by, e.g., detecting and/or measuring RNA expression levels or protein expression levels. For example, RNA levels are detected by quantitative microarray expression analysis, Northern blot analysis, or real time-polymerase chain reaction (RT-PCR). Selective probes for Northern blot analysis or microarray analysis and selective primers for performing quantitative RT-PCR can be identified by examination of the nucleic acid sequences for PDX1 and/or P2RY1 (or members of the CEA family (e.g. CEACAM8, CEACAM6), and/or MSLN). Nucleic acid sequences for PDX1, P2RY1, members of the CEA family such as CEACAM8 and CEACAM6, and MSLN can be found in the art, for example, in the National Center for Biotechnology Information (NCBI) database (www.ncbi.nlm.nih.gov), at GI accession numbers 189095257 and 937547786 and in SEQ ID NOs: 2, 4, 6, 8, and 10.

PDX1 and/or P2RY1 protein (and optionally a protein of the CEA family (e.g. CEACAM8, CEACAM6), and/or MSLN protein) may be detected in the sample of cells. Methods of detecting and quantifying a protein of interest are routine in the art. The amino acid sequence of P2RY1, PDX1, CEACAM8, CEACAM6, and MSLN are provided herein as SEQ ID NOs: 1, 3, 5, 7, and 9. For example, Western blotting and/or enzyme-linked immunosorbent assay (ELISA) are suitable assays for detecting protein expression levels of PDX1, P2RY1, members of the CEA family (e.g. CEACAM8, CEACAM6), and/or MSLN.

The cells express PDX1 and P2RY1. In some embodiments, the cells overexpress PDX1 and/or P2RY1, i.e., one or both proteins are expressed in greater amounts than normally found in healthy, wild-type counterpart cells. The cells in various aspects express members of the CEA family (e.g., CEACAM8, CEACAM6) and/or MSLN. In some embodiments, the cells overexpress members of the CEA family (e.g., CEACAM8, CEACAM6) and/or MSLN.

### Methods, Administration, Compositions

The disclosure provides a method of delivering an agent to a cell expressing PDX1. The cell is a mammalian (e.g., human) cell. Optionally, the cell is a pancreatic cancer cell, a pancreatic cell, a pancreatic progenitor cell, an islet beta cell, or a pancreatic duct cell. In other aspects, the disclosure provides a method treating cancer, such as pancreatic cancer, in a subject. The subject is a mammal (e.g., mouse, rat, rabbit, bird, guinea pig, dog, pig, or cat), preferably a human.

In some embodiments, the method described herein is performed to selectively destruct cancer cells (e.g., pancreatic cancer cells) which express PDX1, using P2RY1 as a surrogate marker for targeting PDX1-expressing cancer cells for destruction. For example, the disclosure provides a method of treating cancer (e.g., pancreatic cancer) in a subject, the method comprising administering to the subject a targeting moiety as described herein and a cytotoxic agent that couples to the targeting moiety. “Couples” refers to any means of attachment of an agent to the targeting moiety, such as those means described herein, e.g., avidin-biotin attachment, antibody-antigen binding, and the like. “Cancer” refers to malignant neoplasms in which cells proliferate uncontrollably, at times forming malignant tumors, which can invade adjacent parts of the body. Cancer may also spread or metastasize to more distant parts of the body through the lymphatic system or bloodstream. The methods of the disclosure are suitable for imaging, diagnosing, treating, preventing spread, monitoring, etc., any type of cancer, tumor, or neoplastic cell that express PDX1 using P2RY1 as a surrogate marker, which was found to be expressed in, e.g., PDX1+ cells of pancreatic adenocarcinoma. A range of cancers are contemplated as targets in the context of the instant method, including, but not limited to, breast cancer, carcinoid, cervical cancer, colorectal cancer, endometrial cancer, glioma, head and neck cancer, liver cancer, lung cancer, lymphoma, skin cancer (including melanoma and non-melanoma), ovarian cancer, pancreatic cancer, prostate cancer, renal cancer, stomach cancer, cancer of the testis, thyroid cancer, and urothelial cancer. Examples of pancreatic cancers include, e.g., pancreatic adenocarcinoma, acinar cell carcinoma, intraductal papillary mucinous neoplasm (IPMN), mucinous cystic neoplasm (MCN), pancreatoblastoma, serous cystadenocarcinoma, solid pseudopapillary, gastrinomas, glucagonomas, insulinomas, somatostatinomas, and VlPomas. While the disclosure discusses cancer cells as target cells, it will be appreciated that the methods described herein extend to any cell type that expresses PDX1 and P2RY1 (including non-cancer cells).

The method of the disclosure provides an efficient and safe regimen for delivering cytotoxic agents to a human for the prophylactic or therapeutic treatment of disease. Ideally, in a subject suffering from cancer, the method inhibits cancer cell proliferation, eradicates cancer cells, and/or reduces the size of at least one tumor. By “treating cancer” is meant alleviation of cancer in whole or in part. The progress of the inventive method in treating cancer (e.g., reducing tumor size or eradicating cancerous cells) is ascertained using any suitable method, such as those methods currently used in the clinic to track tumor size and cancer progress. A primary efficacy parameter for evaluating cancer therapy is a reduction in the size of a tumor or reduction in tumor burden, and the method described herein preferably reduces tumor size and/or burden in the subject. In the context of pancreatic cancer, the method of the disclosure may reduce tumor size, confine a tumor, and/or inhibit spread outside the pancreas (e.g., inhibits or slows spread to lymph nodes, distant sites, and/or blood vessels or nerves). A number of secondary parameters can be employed to further monitor therapy. Examples of secondary parameters include, but are not limited to, detection of new tumors, detection of tumor antigens or markers (e.g., CEA, PSA, or CA-125), biopsy, surgical downstaging (i.e., conversion of the surgical stage of a tumor from unresectable to resectable), PET scans, survival, disease progression-free survival, time to disease progression, quality of life assessments such as the Clinical Benefit Response Assessment, and the like, all of which can point to the overall progression (or regression) of cancer in a human.

The cell is contacted in vitro or in vivo with (i) the targeting moiety that binds P2RY1 and (ii) the agent (optionally attached to a binding moiety that binds the targeting moiety). In some aspects, the cell is further contacted in vitro or in vivo with (iii) a targeting moiety that binds MSLN and/or a targeting moiety that binds CEACAM8 and/or another member of the CEA family, such as CEACAM6. All routes suitable for in vivo administration to a subject are contemplated. For example, the targeting moiety (or targeting moieties) and/or agent may be applied or introduced into body cavities, absorbed through the skin or mucous membranes, ingested, inhaled, and/or introduced into circulation. In various embodiments, the targeting moiety (or targeting moieties) and/or agent is administered via injection or infusion by intravenous, intratumoral, intraperitoneal, intracerebral (intra-parenchymal), intracerebroventricular, intramuscular, intra-ocular, intraarterial, intraportal, intralesional, intramedullary, intrathecal, intraventricular, subcutaneous, intraperitoneal, or enteral means. Transdermal, intranasal, topical, sublingual, urethral, vaginal, or rectal administration is also contemplated. If desired, the targeting moiety (or targeting moieties) and/or agent is administered by controlled, delayed, sustained or otherwise modified release systems, or by implantation devices (e.g., matrix, membrane, sponge, or another appropriate material into which the desired reagent mixture has been absorbed or encapsulated).

In some embodiments, the accumulation of the targeting moieties in the pancreas is facilitated by intrapancreatic artery or intrapancreatic duct delivery. Delivery to the pancreas is optionally facilitated using angiography-assisted intra-pancreatic artery infusion or endoscopic retrograde cholangiopancreatography-assisted intra-pancreatic duct infusion.

The timing of administration of the targeting moiety (or targeting moieties) and agent to the subject, or application to cells, will depend on the particular targeting moiety and agent or agent-binding moiety complex employed. In some embodiments, the targeting moiety binding P2RY1 is conjugated to the agent and, as such, both components are administered or applied simultaneously. Similarly, in some instances the agent comprises a binding moiety, which is bound to the targeting moiety binding P2RY1 (and optionally a targeting moiety that binds MSLN and/or a targeting moiety that binds a member of the CEA family, such as CEACAM8 or CEACAM6) prior to administration or application, resulting in simultaneous administration. In these instances, the targeting moiety and agent-binding moiety complex are presented to the practitioner in the same composition, or as separate compositions which the practitioner combines prior to administration or application. It will be appreciate that the step of combining a targeting moiety and an agent-binding moiety complex may take several minutes (or hours) to allow the binding moieties to join with the targeting moieties.

Alternatively, the (i) targeting moiety and (ii) agent (and, optionally, (iii) additional targeting moieties that bind MSLN or a member of the CEA family, such as CEACAM8 or CEACAM6) are administered or applied to the cells in separate compositions. In this respect, the (i) targeting moiety and (ii) agent (or agent-binding moiety complex) are administered concurrently (near in time). Concurrent administration of the targeting moiety and agent does not require that they be administered at the same time or by the same route. In this aspect, the (i) targeting moiety is administered first, and the (ii) agent (or agent-binding moiety complex) is administered later in time, although an administration regime wherein the (ii) agent (or agent-binding moiety complex) is administered first and the (i) targeting moiety is administered later in time as is contemplated. In various aspects, (iii) a targeting moiety that binds MSLN and/or a targeting moiety that binds a member of the CEA family (e.g., CEACAM8 or CEACAM6) is administered with (concurrently) the targeting moiety that binds P2RY1 in the same or different composition, although this is not required. The time between administration of (i) and (ii) is, in various embodiments, minutes, hours, or days. The time between administrations is, in some instances, a period of time suitable for clearance of free targeting moiety (i.e., targeting moiety not bound to P2RY1) conjugates and/or for non-target cell turnover. The agent (e.g., imaging agent or cytotoxic agent) is then administered, resulting in selective exposure of the agent to targeted cells. In various embodiments, the time between administrations is at least one hour, such as at least three hours, at least six hours, at least eight hours, at least 12 hours, or at least 18 hours. Alternatively, the time between administrations is at least one day (e.g., at least two days, at least three days, at least four days, at least five days, or at least six days) or at least one week. In various aspects, the time between administrations does not exceed one month.

Thus, in one aspect, the disclosure provides a method of treating pancreatic cancer in a subject. The method comprises separately administering to the subject (i) a targeting moiety that binds P2RY1 conjugated to an oxidized variant of avidin and (ii) a cytotoxic agent that couples to the targeting moiety and is conjugated to biotin. Optionally, the method further comprises administering (iii) a targeting moiety that binds MSLN and/or a targeting moiety that binds a member of the CEA family (e.g., CEACAM8 or CEACAM6), which is conjugated to avidin. Optionally, (i) the targeting moiety that binds P2RY1 conjugated to biotin and (ii) the cytotoxic agent that couples to the targeting moiety and is conjugated avidin, such as an oxidized variant of avidin. If the method further comprises administering (iii) a targeting moiety that binds MSLN and/or a targeting moiety that binds a member of the CEA family (e.g., CEACAM8 or CEACAM6), the additional targeting moiety (iii) is conjugated to biotin. The time period between the administration of (i) and (ii) is at least one day.

The amount (dose) of (i) targeting moiety that binds P2RY1 and/or (ii) agent (or agent-binding moiety complex) (and, optionally, (iii) targeting agent that binds a member of the CEA family (e.g., CEACAM8 or CEACAM6) or MSLN) applied to cells or administered to a subject will depend on, e.g., route of administration (local vs. systemic); patient characteristics (e.g., gender, weight, health, side effects); the nature and extent of any medical condition suffered by the subject; and the agent and targeting moiety selected. In general, doses of targeting moieties and binding moieties employed for adult human treatment may be in the range of 100 μg/kg to about 10 mg/kg. The desired dose of (i) targeting moiety and/or (ii) agent (or agent-binding moiety complex) (and, in various aspects, (iii) additional targeting moiety) may be administered in a single dose (i.e., in a single application), or as multiple divided doses. A treatment regimen may comprise multiple rounds of administrations over an extended period of time (e.g., three months, six months, nine months, or more).

The targeting moiety and agent disclosed herein are optionally formulated with pharmaceutically acceptable (e.g., sterile) carriers, providing a pharmaceutical composition suitable for contacting viable cells and administration to a subject (e.g., human). Pharmaceutically acceptable carriers include solvents, dispersion media, coatings, antibacterial and antifungal agents, isotonic and absorption delaying agents, and the like, compatible with pharmaceutical administration.

The disclosure further provides a kit comprising a targeting moiety (or targeting moieties) and an agent (optionally comprising a binding moiety) as described herein. In some embodiments, the targeting moiety and agent are provided in pharmaceutical compositions, either together in a single composition or as separate compositions. Such kits may be used for, e.g., diagnosis and treatment of cancer associated with P2RY1/PDX1 expression in a subject. The kit further comprises instructions for use.

All publications, patents and patent applications cited in this specification are herein incorporated by reference as if each individual publication or patent application were specifically and individually indicated to be incorporated by reference.

What is claimed is:

1. A method of delivering an agent to a cell expressing Pancreatic and Duodenum Homeobox 1 (PDX1) comprising contacting the cell with (i) a targeting moiety that binds Purinergic Receptor P2Y G-Protein Coupled 1 (P2RY1) and (ii) an agent comprising a binding moiety that binds the targeting moiety.

2. The method of claim 1, wherein the targeting moiety is an anti-P2RY1 antibody or antigen-binding fragment thereof, aptamer, or P2RY1 ligand.

3. The method of claim 2, wherein the targeting moiety is an anti-P2RY1 antibody or antigen-binding fragment thereof conjugated to streptavidin.

4. The method of claim 3, wherein the targeting moiety is an anti-P2RY1 antibody or antigen-binding fragment thereof conjugated to an oxidized variant of avidin.

5. The method of claim 2, wherein the targeting moiety is an anti-P2RY1 antibody or antigen-binding fragment thereof conjugated to biotin.

6. The method of claim 5, wherein (ii) is an unsaturated complex of (a) an oxidized variant of avidin and (b) ⁹⁰Y-DOTA-biotin.

7. The method of claim 5, wherein (ii) is an unsaturated complex of (a) an oxidized variant of avidin and (b) ¹⁸F-biotin.

8. The method of any one of claims 1-7, comprising administering (i) and subsequently administering (ii) to the subject.

9. The method of claim 8, wherein the time period between administering (i) and (ii) is at least one day.

10. The method of any one of claims 1-9, further comprising contacting the cell with (iii) a targeting moiety that binds a member of the carcinoembryonic antigen (CEA) family or a targeting moiety that binds mesothelin (MSLN).

11. The method of claim 10, wherein the member of the CEA family is carcinoembryonic antigen-related cell adhesion molecule 8 (CEACAM8) or carcinoembryonic antigen-related cell adhesion molecule 6 (CEACAM6).

12. The method of claim 10 or claim 11, wherein the targeting moiety that binds a member of the CEA family or the targeting moiety that binds MSLN is an antibody or antigen-binding fragment thereof, aptamer, or ligand.

13. The method of claim 12, wherein the targeting moiety that binds a member of the CEA family or the targeting moiety that binds MSLN is an antibody or antigen-binding fragment thereof conjugated to biotin.

14. A method of delivering an agent to a cell expressing Pancreatic and Duodenum Homeobox 1 (PDX1) comprising contacting the cell with a targeting moiety that binds Purinergic Receptor P2Y G-Protein Coupled 1 (P2RY1) conjugated to the agent.

15. The method of claim 14 wherein the targeting moiety is an anti-P2RY1 antibody or antigen-binding fragment thereof, aptamer, or P2RY1 ligand.

16. The method of claim 14 or claim 15, wherein the agent is further conjugated to a targeting moiety that binds a member of the CEA family, a targeting moiety that binds mesothelin (MSLN), or a combination thereof.

17. The method of claim 16, wherein the member of the CEA family is CEACAM8 or CEACAM6.

18. The method of claim 17, wherein the targeting moiety that binds the member of the CEA family or the targeting moiety that binds MSLN is an antibody or antigen-binding fragment thereof, aptamer, or ligand.

19. The method of any one of claims 1-18, wherein the cell is a pancreatic cancer cell, an islet beta cell, a pancreatic duct cell, or a pancreatic progenitor cell.

20. The method of any one of claims 1-5 and 8-19, wherein the agent is a cytotoxic agent, an immune mediator, a growth factor, an immunosuppressant, a fluorochrome, a radioactive substance, a metal, a hormone, an extracellular matrix component, a cell-adhesion molecule, biomaterial, or an imaging reagent.

21. The method of any one of claims 1-20, wherein, prior to the contacting step, the method comprises determining expression of PDX1 in a sample of the cells.

22. The method of any one of claims 1-20, wherein the cell is contacted in vivo and the targeting moiety that binds P2RY1 and agent are delivered intravenously.

23. The method of any one of claims 1-20, wherein the cell is contacted in vivo and the targeting moiety that binds P2RY1 and agent are delivered by intra-pancreatic artery infusion or intra-pancreatic duct infusion.

24. A method of treating pancreatic cancer in a subject, the method comprising administering to the subject (i) a targeting moiety that binds Purinergic Receptor P2Y G-Protein Coupled 1 (P2RY1) and (ii) a cytotoxic agent that couples to the targeting moiety.

25. The method of claim 24, wherein administration of (i) and (ii) reduces tumor size or tumor burden in the subject.

26. The method of claim 24 or claim 25, further comprising contacting the cell with (iii) a targeting moiety that binds a member of the CEA family or a targeting moiety that binds mesothelin (MSLN).

27. The method of claim 26, wherein the member of the CEA family is CEACAM8 or CEACAM6.

28. A method of detecting cells expressing Pancreatic and Duodenum Homeobox 1 (PDX1) in vivo, the method comprising
(a) administering to the subject (i) a targeting moiety that binds Purinergic Receptor P2Y G-Protein Coupled 1 (P2RY1) and (ii) an imaging agent that couples to the targeting moiety, and
(b) detecting the imaging agent.

29. The method of any one of claims 24-29, wherein the agent is coupled to the targeting moiety that binds P2RY1 prior to administration to the subject.

30. The method of claim 29, wherein the agent is further coupled to a targeting moiety that binds a member of the CEA family or a targeting moiety that binds MSLN.

31. The method of claim 30, wherein the member of the CEA family is CEACAM8 or CEACAM6.

32. The method of any one of claims 24-28, wherein the agent is not coupled to the targeting moiety prior to administration, and the agent comprises a binding moiety that binds the targeting moiety.

33. The method of claim 32, comprising administering (i) and subsequently administering (ii) to the subject.

34. The method of claim 34, wherein the time period between administering (i) and (ii) is at least one day.

35. The method of any one of claims 24-34, wherein the targeting moiety that binds P2RY1 is an anti-P2RY1 antibody or antigen-binding fragment thereof, aptamer, or P2RY1 ligand.

36. The method of claim 35, wherein the targeting moiety that binds P2RY1 is an anti-P2RY1 antibody or antigen-binding fragment thereof conjugated to streptavidin.

37. The method of claim 35, wherein the targeting moiety that binds P2RY1 is an anti-P2RY1 antibody or antigen-binding fragment thereof conjugated to an oxidized variant of avidin.

38. The method of claim 35, wherein the targeting moiety that binds P2RY1 is an anti-P2RY1 antibody or antigen-binding fragment thereof conjugated to biotin.

39. The method of claim 24, wherein (i) is an anti-P2RY1 antibody or antigen-binding fragment thereof conjugated to biotin and (ii) is an unsaturated complex of (a) an oxidized variant of avidin and (b) ⁹⁰Y-DOTA-biotin.

40. The method of claim 28, wherein (i) is an anti-P2RY1 antibody or antigen-binding fragment thereof conjugated to biotin and (ii) is an unsaturated complex of (a) an oxidized variant of avidin and (b) ¹⁸F-biotin.

41. The method of any one of claims 24-40, wherein (i) and (ii) are delivered intravenously.

42. The method of any one of claims 24-40, wherein (i) and (ii) are delivered by intra-pancreatic artery infusion or intra-pancreatic duct infusion.

43. A method of treating pancreatic cancer in a subject, the method comprising separately administering to the subject (i) a targeting moiety that binds Purinergic Receptor P2Y G-Protein Coupled 1 (P2RY1) conjugated to an oxidized variant of avidin and (ii) a cytotoxic agent that couples to the targeting moiety and is conjugated to biotin, wherein the time period between the administration of (i) and (ii) is at least one day.

44. The method of claim 39, wherein (iii) a targeting moiety that binds a member of the CEA family or a targeting moiety that binds mesothelin (MSLN) also is conjugated to the oxidized variant of avidin.

45. The method of claim 44, wherein the member of the CEA family is CEACAM8 or CEACAM6.


SEQUENCE LISTING (10 sequences)

SEQ ID NO: 1 - protein, 372 aa, Homo sapiens
  MISC_FEATURE: P2Y1
TEVLWPAVPNGTDAAFLAGPGSSWGNSTVASTAAVSSSFKCALTKTGFQFYYLPAVYILVFIIGFLGNSVAIWMFVFHMKPWSGISVYMFNLALADFLYVLTLPALIFYYFNKTDWIFGDAMCKLQRFIFHVNLYGSILFLTCISAHRYSGVVYPLKSLGRLKKKNAICISVLVWLIVVVAISPILFYSGTGVRKNKTITCYDTTSDEYLRSYFIYSMCTTVAMFCVPLVLILGCYGLIVRALIYKDLDNSPLRRKSIYLVIIVLTVFAVSYIPFHVMKTMNLRARLDFQTPAMCAFNDRVYATYQVTRGLASLNSCVDPILYFLAGDTFRRRLSRATRKASRRSEANLQSKSEDMTLNILPEFKQNGDTSL

SEQ ID NO: 2 - DNA, 6802 bp, Homo sapiens
  misc_feature: P2Y1
acctggcgtggttctcgagggccctagaggctcctgctacggactggtcggctctggggttgtaggcagaaggctctaagcggtcggaagggtttcctgtgtgtcagcaacttcactttgccgagatagctggcggaagagggaaggagcgctcgcgggccaatcgcctgccagcagccaccttggagcgcacccctctccctcgccgccgactccggtagcagcgtgccggcagaggctgaacacactcggcctcggcggagacctgctccccagaagacgcctcctgcttcccactgcgccctggaggacgcgggctggctgctgggcgagctcggcggaggcacgcccctcgcctccccgcggagtgcggactcgccccggtgcccaaactccgcccaccctctagggagctccgctctcccgcctaaccccggcactccggacagagctgggcctggggaaggggttcctgaactacgcggacgccgaacgggacgcgctgcagaagcgcacgagtctgcggccacgcgcgctccgatggctgccaggagctgagctcagggtgggcggaggaagcggttagacgccccgaaactgagctgcacgtttctaaggtagggaggaggaagatgcccccaattaagttgatctttgagccaaggaggctggggagcagcctccccaagctagagccctgcagagcgagtttcccttgacctcgctgcgcctctggcgcgctctgcagcgcggacccgcggcccctcgggaaagcgcagtcggaaagttatccgcggcggttccctgcgcgccctgttgtgtaagctcggcgttgccagcggacggagaagttgctggcttgcccgatagcccagttcggtggcggcccggggcggatttcatggcccgcggcgaacgcggggccagagctggcgtgggcgagcccctgcgcgccccctcccgcggggatccagttcgcctgctcccttccgctcgctggcttttccgatgcttgctgcgcccctggccgccgctgccctctcgccgcctcctacccctcggagccgccgcctaagtcgaggaggagagaatgaccgaggtgctgtggccggctgtccccaacgggacggacgctgccttcctggccggtccgggttcgtcctgggggaacagcacggtcgcctccactgccgccgtctcctcgtcgttcaaatgcgccttgaccaagacgggcttccagttttactacctgccggctgtctacatcttggtattcatcatcggcttcctgggcaacagcgtggccatctggatgttcgtcttccacatgaagccctggagcggcatctccgtgtacatgttcaatttggctctggccgacttcttgtacgtgctgactctgccagccctgatcttctactacttcaataaaacagactggatcttcggggatgccatgtgtaaactgcagaggttcatctttcatgtgaacctctatggcagcatcttgtttctgacatgcatcagtgcccaccggtacagcggtgtggtgtaccccctcaagtccctgggccggctcaaaaagaagaatgcgatctgtatcagcgtgctggtgtggctcattgtggtggtggcgatctcccccatcctcttctactcaggtaccggggtccgcaaaaacaaaaccatcacctgttacgacaccacctcagacgagtacctgcgaagttatttcatctacagcatgtgcacgaccgtggccatgttctgtgtccccttggtgctgattctgggctgttacggattaattgtgagagctttgatttacaaagatctggacaactctcctctgaggagaaaatcgatttacctggtaatcattgtactgactgtttttgctgtgtcttacatccctttccatgtgatgaaaacgatgaacttgagggcccggcttgattttcagaccccagcaatgtgtgctttcaatgacagggtttatgccacgtatcaggtgacaagaggtctagcaagtctcaacagttgtgtggaccccattctctatttcttggcgggagatactttcagaaggagactctcccgagccacaaggaaagcttctagaagaagtgaggcaaatttgcaatccaagagtgaagacatgaccctcaatattttacctgagttcaagcagaatggagatacaagcctgtgaaggcacaagaatctccaaacacctctctgttgtaatatggtaggatgcttaacagaatcaagtacttttcccctctttaactttctagtttagaaaaaaatcaaaccaagaaaatagtgagttaaaaaaataatagaagtagaaatgcccacatccacacttagcttgtttgggtttgctttcacagtctctcttccttctgactagaagtatgtataataaaacaatactacctagttaaacatttactttctcttttgcctttaaaatgtgcaggcttttctgtttaaagtgtgtgtgcacatgagtactggggctgtttttgatattagtaatttctctaagaaaactagccccctgcaacttgagtttgtggtttatctagcctttattgtttttttaaaatccacagtaggaataaaaaatctatattctcagaaatatctagcatggtatataacaaaacactaaactcatcagttcatccggcatcagatcaatggatctctgagcggggtgtttttttcagtgtcttataagcatagatgatagttgactgagtttctttagggcattgaatagacaagtaaagctaatgaatttaaaagcctgaaaagtgattgttttccagttatttctggaaaaggtctcattatatattgggtgctaaatgtttgatggggaaagcctgcatatattatcgtactggtaaaatgcattcaaaataattaaagtgcatgtattttccttgtaaacaccatgagctctcttagacatcttgtgataaagagcatttacttgccccactgctgtgcaatgccttaggactttgtttgtgttccaggacaagtgttcactcacatctgtaaaaacaattttaagaattgcaaataaattacagaccaaagattgagtaaagtcaaataactgttagtaagttgaaggatattggacaggaggacagtatttcagaaaaggagaggttgacagtcatccacaaggcatagcctccaagtatactctcaaatgtatgaagcaactggggtgggcagaagacattttagaatgagggctttagtttaaattaaagtcatggtggagaagactcttgcttcctccaagtgtttgaaaacacaaaatgcgatatgaaaagaagaaatagaatttgtataggttgcattgatagaatgggaagctataatctttgaataaaccttacatctcaatatttaaagaaaaaacatatatatggagggaaatatagagaattaaagtacctagatgccatagcactgaaaagtaaagtactactcaaactactaatagaagactattggtcagtgttttctattatatttgttctctgccaggtgttaatgaaaaatttgtaaaatgcttcaacagtgttaataattttaatgaatattgaattgctgcatgtctagtccttgaaatggtcaaattctatataatgagaataacaaggtgagataatgcaattgacaatcccaaacaaatccagtgtcaaaggaatttttagttttggaagctatccctcataaagtcaacatcagagtttgcaactgatggtggcataacttgtttgaaagcactttacaagtagagcaacatggggctgtatgttaaggcaagaaagaaccttagtgccccgtggaccataatgacaggaaattttcaggtcttgatttcagttcttgcaatgcctatgtagattgaggatttgtgagaccttcagagtagcttcactttacccaatctctggccactaagatcaatgaagaaaagctgtatttaaccttccaaagcagtgattctttttacatgttcagttttaatgcataggattatagaaaccaaatcaagaagaaacctactacacctccttattttgaggaaagagagggttagcaaagttctcacttcatcaggctcaaataacatccgttttagattcctacaagacaaaatctaaaatatgtcattactttattgaccttgttaaacaagatcaatatcattaaactgaccaagatggaaattacaggacttgtaccagacttgaacttgtagcctaatgatacacatttttacaatatttcttcaccatttagttttctttcttttaggtctcaggatgctcagcttaatttagagtatgtgttaagtacattgtttcagcgtccagagaattttgtgagtgtggatccagtgaggaggtgaacagtaactagaaatgcttgtttactacttggcatgttttttccaaagtgtttcttctttagcttcatacatcacatatggacactgatgacatgtgaatgcatgccacatagtaacaagaatgatcttgccaactccttccaagccaaaagaagcctctgggaaggtgtgccaatggtgaaaacgtacagagggtcagggtgaagcagtgatctctattggctgaaaaactgatcaggtcatatgatgattgaagtatgtttattgtaagggcagaaatgtgttggcatttggataaaaaactgctaacattatagaacttattacctaacaaaatttcacaccacaaaaaatattttaatggcaaattcaaggtgttttattgcttacaaatcagcatctttgactctttgaacatcaatttgtgtttacattgaaatgacaaaaagacaaactaagaagaaatacagcatgcaagttggaattcagagttaaaaccatgatgttgccgctcagccagctatgtgactgttgaccctttcaagaacacacatggatttaaaagttggatgacatccattgttggggccttgggggatatggtaaagcatgaaaactaaacagccaggagcctgtgaaatctgctactgtattttccaggacttcattccactccttggctaaaaaaatcttggaagtttcacagattatgatgtggacctgtcacctgtaaattgtctcaatctactcagacaagacactaaactgtctttggatactatagatgtcagtgcttatagcagctggaatttggctagtgacaatgtttaaagatgtaatactagttagtatctattgaagcttaaactttgctggtcaggttgtagctattgtaaaagtatttattgaagaagctcacagtccttcagttgtacagactgaaaaactttcatgaaagatccaacatactaatgtaaattatatttattacaatgtatgatattaatgtgtcaaactggtgtattttacaaaatatataatgcatacataaataagagttgtatattacagtgcttttcaaatatcagtgtcttggaatatttaagtcttcacattttttggtctaaaatatgaaaatgtttcatgatacaagtgattaattttccctagtagtgcttttgcatgtttgcctttttatttaagtttttttctatatagacacaatttggtgtcagactatcataagatcgatagtgaatataaaatatcttagccaaatggggtctgtattgtctacattttatatattaaataaaagtttttgttgtcttttcaggaggtttagagtattgtcactaaatatgatcaaagcttccctttccaaatgcaaaagtcttgtcctacatttaaagttgatctgtcatgttttagcagtcaagtgggatgggcattatataaacaacgttacaatgtaaggaaaatctttaaggagatggggagagaaaaaggcagctggtataatcggttactgctgcttagttctacttaattttttgtgttgcttcttcttaaggtgagatagcataatcttaactgttttgagatggaattttaaagtaacacactaccagcgagttcaacactgctattgattttaatctgtttttttttgtttagttgataacttaaattccaagtttcatagtgataattgtatattatttggctgctgaattctgttagagttttttattctgttgtacattgtattatacacataatcacaaattaatatgaaggtgaatatatgttacatatcaaaatttgtgaatttgaattatagtatgttttagtgctattgcaaaaaatgtttatttttatattatctgtgattttaatatagatgattgaactagatttctttttgagtgatagtgccattgaatgagcagtatggaaacagtgttacttgatattttgagctttctcaggtttatctaaatcagtggtagcttaacaaaacccagactaattgtgtgtaattgtatttttaataaaaggaaagtacatttcctataatagcatagtactgtttgcatgtaagagtatgcaaaaccttgtgtgtgtgtgtgtgtgtgtgtgtgtgtgtgtgtcttagtgtgtgtaaggcatggcagccaactttgtatctgctatttttagtacgagcagagcttcataattgtggtcactagaactgtacttaccatggacagttaaaactgaaaaagactcaataaaactatgaaacatggaaaaaaaaaaaaaaaaaaaaaaaaaaaaaaaaaaaaaaaaaaaaaaaaaaaaaaa

SEQ ID NO: 3 - protein, 349 aa, Homo sapiens
  MISC_FEATURE: CEACAM8
MGPISAPSCRWRIPWQGLLLTASLFTFWNPPTTAQLTIEAVPSNAAEGKEVLLLVHNLPQDPRGYNWYKGETVDANRRIIGYVISNQQITPGPAYSNRETIYPNASLLMRNVTRNDTGSYTLQVIKLNLMSEEVTGQFSVHPETPKPSISSNNSNPVEDKDAVAFTCEPETQNTTYLWWVNGQSLPVSPRLQLSNGNRTLTLLSVTRNDVGPYECEIQNPASANFSDPVTLNVLYGPDAPTISPSDTYYHAGVNLNLSCHAASNPPSQYSWSVNGTFQQYTQKLFIPNITTKNSGSYACHTTNSATGRNRTTVRMITVSDALVQGSSPGLSARATVSIMIGVLARVALI

SEQ ID NO: 4 - DNA, 2333 bp, Homo sapiens
  misc_feature: CEACAM8
agcacaaaaggaggaaggacagcacagctgacagccgtgctcagaaagtttctggatcccaggctcatctccacagaggagaacacgcaggcagcagagaccatggggcccatctcagccccttcctgcagatggcgcatcccctggcaggggctcctgctcacagcctcacttttcaccttctggaacccgcccaccactgctcagctcactattgaagctgtgccatccaatgctgcagaggggaaggaggttcttctacttgtccacaatctgccccaggaccctcgtggctacaactggtacaaaggggaaacagtggatgccaaccgtcgaattataggatatgtaatatcaaatcaacagattaccccagggcctgcatacagcaatcgagagacaatataccccaatgcatccctgctgatgcggaacgtcaccagaaatgacacaggatcctacaccctacaagtcataaagctaaatcttatgagtgaagaagtaactggccagttcagcgtacatccggagactcccaagccctccatctccagcaacaactccaaccccgtggaggacaaggatgctgtggccttcacctgtgaacctgagactcagaacacaacctacctgtggtgggtaaatggtcagagtctcccggtcagtcccaggctgcagctgtccaatggcaacaggaccctcactctactcagtgtcacaaggaatgacgtaggaccctatgaatgtgaaatacagaacccagcgagtgcaaacttcagtgacccagtcaccctgaatgtcctctatggcccagatgcccccaccatttccccttcagacacctattaccatgcaggggtaaatctcaacctctcctgccatgcggcctctaatccaccctcacagtattcttggtctgtcaatggcacattccagcaatacacacaaaagctctttatccccaacatcactacaaagaacagcggatcctatgcctgccacaccactaactcagccactggccgcaacaggaccacagtcaggatgatcacagtctctgatgctttagtacaaggaagttctcctggcctctcagctagagccactgtcagcatcatgattggagtactggccagggtggctctgatatagtagctctggtgtagtttctgcatttcaagaagactggcagacagttgtttttattcttcctcaaagcatttgcaatcagctaccattcaaaattgcttcttcttcaagatttatggaaaatactctgacgagtactcttgaacacaagttcctgataactttaagatcacgccactggactgtctatgaacttgcaaacaggctgatacctttgtgaagttgcccaccaaaacacagaaggaaaaaaacatgaatttcattgaactaaataataatgaggataatgtttttaagatttttttttttttttttttgagatggaatctcgctctgtcgcccaggctggagtgcagtggcacgatctcaactcactgcaagctccgcctcctgggttcacaccattctcctgcctcagcctcctgagtagctgggactacaggcgcctgccacaacgcccggctaattttttgtatttttagtagagacggggtttcactgtggtctcaatctcctgacttcatggtccgcctgcctcagcctcccaaagttctgggattacaggtgtgagccaccgcgcccagcccgtttttaagattttttatttgaaaaattgccaattctttaagtgttttctttttcagatttatgaatttctttatcttttaagctatctataccttactgcaatttggtaaagcagacttttgtgaacaaaaattataacatttacttttgctccctacctgactgccacagaactgggcaactattcatgagtattcatatgtttatggtaattcagttatttgcacaagttcagtgagaatctgctgtctttataatgggatatagtttaaaacattggttatattaccaaggctttgattgggatgttatatttgagaaaatacagagaatgatagattaacggagtgtctaatctatcgtgtcaaccccaaatttttacgtatgagatcctttagtccacccaatggctgacagtaacagcatctttaacacaactctttgttcaaatgtactatggtctcttttagagtcagactcctagactcacttgttctcactgtctgttttaatttaacccaggcatgcaatgctagataataaaattgctccctattggctgatcaaaaaaaaaaaaaaaaaaaaaaaaaaaaaaa

SEQ ID NO: 5 - protein, 344 aa, Homo sapiens
  MISC_FEATURE: CEACAM6
MGPPSAPPCRLHVPWKEVLLTASLLTFWNPPTTAKLTIESTPFNVAEGKEVLLLAHNLPQNRIGYSWYKGERVDGNSLIVGYVIGTQQATPGPAYSGRETIYPNASLLIQNVTQNDTGFYTLQVIKSDLVNEEATGQFHVYPELPKPSISSNNSNPVEDKDAVAFTCEPEVQNTTYLWWVNGQSLPVSPRLQLSNGNMTLTLLSVKRNDAGSYECEIQNPASANRSDPVTLNVLYGPDGPTISPSKANYRPGENLNLSCHAASNPPAQYSWFINGTFQQSTQELFIPNITVNNSGSYMCQAHNSATGLNRTTVTMITVSGSAPVLSAVATVGITIGVLARVALI

SEQ ID NO: 6 - DNA, 2601 bp, Homo sapiens
  misc_feature: CEACAM6
gaggctcagcacagaaggaggaaggacagcagggccaacagtcacagcagccctgaccagagcattcctggagctcaagctcctctacaaagaggtggacagagaagacagcagagaccatgggacccccctcagcccctccctgcagattgcatgtcccctggaaggaggtcctgctcacagcctcacttctaaccttctggaacccacccaccactgccaagctcactattgaatccacgccgttcaatgtcgcagaggggaaggaggttcttctactcgcccacaacctgccccagaatcgtattggttacagctggtacaaaggcgaaagagtggatggcaacagtctaattgtaggatatgtaataggaactcaacaagctaccccagggcccgcatacagtggtcgagagacaatataccccaatgcatccctgctgatccagaacgtcacccagaatgacacaggattctataccctacaagtcataaagtcagatcttgtgaatgaagaagcaaccggacagttccatgtatacccggagctgcccaagccctccatctccagcaacaactccaaccccgtggaggacaaggatgctgtggccttcacctgtgaacctgaggttcagaacacaacctacctgtggtgggtaaatggtcagagcctcccggtcagtcccaggctgcagctgtccaatggcaacatgaccctcactctactcagcgtcaaaaggaacgatgcaggatcctatgaatgtgaaatacagaacccagcgagtgccaaccgcagtgacccagtcaccctgaatgtcctctatggcccagatgtccccaccatttccccctcaaaggccaattaccgtccaggggaaaatctgaacctctcctgccacgcagcctctaacccacctgcacagtactcttggtttatcaatgggacgttccagcaatccacacaagagctctttatccccaacatcactgtgaataatagcggatcctatatgtgccaagcccataactcagccactggcctcaataggaccacagtcacgatgatcacagtctctggaagtgctcctgtcctctcagctgtggccaccgtcggcatcacgattggagtgctggccagggtggctctgatatagcagccctggtgtattttcgatatttcaggaagactggcagattggaccagaccctgaattcttctagctcctccaatcccattttatcccatggaaccactaaaaacaaggtctgctctgctcctgaagccctatatgctggagatggacaactcaatgaaaatttaaagggaaaaccctcaggcctgaggtgtgtgccactcagagacttcacctaactagagacagtcaaactgcaaaccatggtgagaaattgacgacttcacactatggacagcttttcccaagatgtcaaaacaagactcctcatcatgataaggctcttacccccttttaatttgtccttgcttatgcctgcctctttcgcttggcaggatgatgctgtcattagtatttcacaagaagtagcttcagagggtaacttaacagagtgtcagatctatcttgtcaatcccaacgttttacataaaataagagatcctttagtgcacccagtgactgacattagcagcatctttaacacagccgtgtgttcaaatgtacagtggtccttttcagagttggacttctagactcacctgttctcactccctgttttaattcaacccagccatgcaatgccaaataatagaattgctccctaccagctgaacagggaggagtctgtgcagtttctgacacttgttgttgaacatggctaaatacaatgggtatcgctgagactaagttgtagaaattaacaaatgtgctgcttggttaaaatggctacactcatctgactcattctttattctattttagttggtttgtatcttgcctaaggtgcgtagtccaactcttggtattaccctcctaatagtcatactagtagtcatactccctggtgtagtgtattctctaaaagctttaaatgtctgcatgcagccagccatcaaatagtgaatggtctctctttggctggaattacaaaactcagagaaatgtgtcatcaggagaacatcataacccatgaaggataaaagccccaaatggtggtaactgataatagcactaatgctttaagatttggtcacactctcacctaggtgagcgcattgagccagtggtgctaaatgctacatactccaactgaaatgttaaggaagaagatagatccaattaaaaaaaattaaaaccaatttaaaaaaaaaaagaacacaggagattccagtctacttgagttagcataatacagaagtcccctctactttaacttttacaaaaaagtaacctgaactaatctgatgttaaccaatgtatttatttctgtggttctgtttccttgttccaatttgacaaaacccactgttcttgtattgtattgcccagggggagctatcactgtacttgtagagtggtgctgctttaattcataaatcacaaataaaagccaattagctctataact

SEQ ID NO: 7 - protein, 622 aa, Homo sapiens
  MISC_FEATURE: MSLN
MALPTARPLLGSCGTPALGSLLFLLFSLGWVQPSRTLAGETGQEAAPLDGVLANPPNISSLSPRQLLGFPCAEVSGLSTERVRELAVALAQKNVKLSTEQLRCLAHRLSEPPEDLDALPLDLLLFLNPDAFSGPQACTRFFSRITKANVDLLPRGAPERQRLLPAALACWGVRGSLLSEADVRALGGLACDLPGRFVAESAEVLLPRLVSCPGPLDQDQQEAARAALQGGGPPYGPPSTWSVSTMDALRGLLPVLGQPIIRSIPQGIVAAWRQRSSRDPSWRQPERTILRPRFRREVEKTACPSGKKAREIDESLIFYKKWELEACVDAALLATQMDRVNAIPFTYEQLDVLKHKLDELYPQGYPESVIQHLGYLFLKMSPEDIRKWNVTSLETLKALLEVNKGHEMSPQVATLIDRFVKGRGQLDKDTLDTLTAFYPGYLCSLSPEELSSVPPSSIWAVRPQDLDTCDPRQLDVLYPKARLAFQNMNGSEYFVKIQSFLGGAPTEDLKALSQQNVSMDLATFMKLRTDAVLPLTVAEVQKLLGPHVEGLKAEERHRPVRDWILRQRQDDLDTLGLGLQGGIPNGYLVLDLSMQEALSGTPCLLGPGPVLTVLALLLASTLA

SEQ ID NO: 8 - DNA, 2184 bp, Homo sapiens
  misc_feature: MSLN
tgccaggctctccacccccacttcccaattgaggaaaccgaggcagaggaggctcagcgccacgcactcctctttctgcctggccggccactcccgtctgctgtgacgcgcggacagagagctaccggtggacccacggtgcctccctccctgggatctacacagaccatggccttgccaacggctcgacccctgttggggtcctgtgggacccccgccctcggcagcctcctgttcctgctcttcagcctcggatgggtgcagccctcgaggaccctggctggagagacagggcaggaggctgcgcccctggacggagtcctggccaacccacctaacatttccagcctctcccctcgccaactccttggcttcccgtgtgcggaggtgtccggcctgagcacggagcgtgtccgggagctggctgtggccttggcacagaagaatgtcaagctctcaacagagcagctgcgctgtctggctcaccggctctctgagccccccgaggacctggacgccctcccattggacctgctgctattcctcaacccagatgcgttctcggggccccaggcctgcacccatttcttctcccgcatcacgaaagccaatgtggacctgctcccgaggggggctcccgagcgacagcggctgctgcctgcggctctggcctgctggggtgtgcgggggtctctgctgagcgaggctgatgtgcgggctctgggaggcctggcttgcgacctgcctgggcgctttgtggccgagtcggccgaagtgctgctaccccggctggtgagctgcccgggacccctggaccaggaccagcaggaggcagccagggcggctctgcagggcgggggacccccctacggccccccgtcgacatggtctgtctccacgatggacgctctgcggggcctgctgcccgtgctgggccagcccatcatccgcagcatcccgcagggcatcgtggccgcgtggcggcaacgctcctctcgggacccatcctggcggcagcctgaacggaccatcctccggccgcggttccggcgggaagtggagaagacagcctgtccttcaggcaagaaggcccgcgagatagacgagagcctcatcttctacaagaagtgggagctggaagcctgcgtggatgcggccctgctggccacccagatggaccgcgtgaacgccatccccttcacctacgagcagctggacgtcctaaagcataaactggatgagctctacccacaaggttaccccgagtctgtgatccagcacctgggctacctcttcctcaagatgagccctgaggacattcgcaagtggaatgtgacgtccctggagaccctgaaggctttgcttgaagtcaacaaagggcacgaaatgagtcctcaggtggccaccctgatcgaccgctttgtgaagggaaggggccagctagacaaagacaccctagacaccctgaccgccttctaccctgggtacctgtgctccctcagccccgaggagctgagctccgtgccccccagcagcatctgggcggtcaggccccaggacctggacacgtgtgacccaaggcagctggacgtcctctatcccaaggcccgccttgctttccagaacatgaacgggtccgaatacttcgtgaagatccagtccttcctgggtggggcccccacggaggatttgaaggcgcttagtcagcagaatgtgagcatggacttggccacgttcatgaagctgcggacggatgcggtgctgccgttgactgtggctgaggtgcagaaacttctgggaccccacgtggagggcctgaaggcggaggagcggcaccgcccggtgcgggactggatcctacggcagcggcaggacgacctggacacgctggggctggggctacagggcggcatccccaacggctacctggtcctagacctcagcgtgcaagaggccctctcggggacgccctgcctcctaggacctggacctgttctcaccgtcctggcactgctcctagcctccaccctggcctgagggccccactcccttgctggccccagccctgctggggatccccgcctggccaggagcaggcacgggtggtccctgttccaccccaagagaactcgcgctcagtaaacgggaacatgccccctgcaaaaaaaaaaaaaaaaaaaaaaa

SEQ ID NO: 9 - protein, 283 aa, Homo sapiens
  MISC_FEATURE: PDX1
MNGEEQYYAATQLYKDPCAFQRGPAPEFSASPPACLYMGRQPPPPPPHPFPGALGALEQGSPPDISPYEVPPLADDPAVAHLHHHLPAQLALPHPPAGPFPEGAEPGVLEEPNRVQLPFPWMKSTKAHAWKGQWAGGAYAAEPEENKRTRTAYTRAQLLELEKEFLFNKYISRPRRVELAVMLNLTERHIKIWFQNRRMKWKKEEDKKRGGGTAVGGGGVAEPEQDCAVTSGEELLALPPPPPPGGAVPPAAPVAAREGRLPPGLSASPQPSSVAPRRPQEPR

SEQ ID NO: 10 - DNA, 2573 bp, Homo sapiens
  misc_feature: PDX1
gggtggcgccgggagtgggaacgccacacagtgccaaatccccggctccagctcccgactcccggctcccggctcccggctcccggtgcccaatcccgggccgcagccatgaacggcgaggagcagtactacgcggccacgcagctttacaaggacccatgcgcgttccagcgaggcccggcgccggagttcagcgccagcccccctgcgtgcctgtacatgggccgccagcccccgccgccgccgccgcacccgttccctggcgccctgggcgcgctggagcagggcagccccccggacatctccccgtacgaggtgccccccctcgccgacgaccccgcggtggcgcaccttcaccaccacctcccggctcagctcgcgctcccccacccgcccgccgggcccttcccggagggagccgagccgggcgtcctggaggagcccaaccgcgtccagctgcctttcccatggatgaagtctaccaaagctcacgcgtggaaaggccagtgggcaggcggcgcctacgctgcggagccggaggagaacaagcggacgcgcacggcctacacgcgcgcacagctgctagagctggagaaggagttcctattcaacaagtacatctcacggccgcgccgggtggagctggctgtcatgttgaacttgaccgagagacacatcaagatctggttccaaaaccgccgcatgaagtggaaaaaggaggaggacaagaagcgcggcggcgggacagctgtcgggggtggcggggtcgcggagcctgagcaggactgcgccgtgacctccggcgaggagcttctggcgctgccgccgccgccgccccccggaggtgctgtgccgcccgctgcccccgttgccgcccgagagggccgcctgccgcctggccttagcgcgtcgccacagccctccagcgtcgcgcctcggcggccgcaggaaccacgatgagaggcaggagctgctcctggctgaggggcttcaaccactcgccgaggaggagcagagggcctaggaggaccccgggcgtggaccacccgccctggcagttgaatggggcggcaattgcggggcccaccttagaccgaaggggaaaacccgctctctcaggcgcatgtgccagttggggccccgcgggtagatgccggcaggccttccggaagaaaaagagccattggtttttgtagtattggggccctcttttagtgatactggattggcgttgtttgtggctgttgcgcacatccctgccctcctacagcactccaccttgggacctgtttagagaagccggctcttcaaagacaatggaaactgtaccatacacattggaaggctccctaacacacacagcggggaagctgggccgagtaccttaatctgccataaagccattcttactcgggcgacccctttaagtttagaaataattgaaaggaaatgtttgagttttcaaagatcccgtgaaattgatgccagtggaatacagtgagtcctcctcttcctcctcctcctcttccccctccccttcctcctcctcctcttcttttccctcctcttcctcttcctcctgctctcctttcctccccctcctcttttccctcctcttcctcttcctcctgctctcctttcctccccctcctctttctcctcctcctcctcttcttccccctcctctccctcctcctcttcttccccctcctctccctcctcctcttcttctccctcctcttcctcttcctcctcttccacgtgctctcctttcctccccctcctcttgctccccttcttccccgtcctcttcctcctcctcctcttcttctccctcctcttcctcctcctctttcttcctgacctctttctttctcctcctcctccttctacctccccttctcatccctcctcttcctcttctctagctgcacacttcactactgcacatcttataacttgcacccctttcttctgaggaagagaacatcttgcaaggcagggcgagcagcggcagggctggcttaggagcagtgcaagagtccctgtgctccagttccacactgctggcagggaaggcaaggggggacgggcctggatctgggggtgagggagaaagatggacccctgggtgaccactaaaccaaagatattcggaactttctatttaggatgtggacgtaattcctgttccgaggtagaggctgtgctgaagacaagcacagtggcctggtgcgccttggaaaccaacaactattcacgagccagtatgaccttcacatctttagaaattatgaaaacgtatgtgattggagggtttggaaaaccagttatcttatttaacattttaaaaattacctaacagttatttacaaacaggtctgtgcatcccaggtctgtcttcttttcaaggtctgggccttgtgctcgggttatgtttgtgggaaatgcttaataaatactgataatatgggaagagatgaaaactgattctcctcactttgtttcaaacctttctggcagtgggatgattcgaattcacttttaaaattaaattagcgtgttttgttttg